SEQUENCE LISTING (16 sequences)

SEQ ID NO: 1 - DNA, 1017 bp, Homo sapiens
cccttggccagcgagatgctttgacacagccccttaagatgttttaagggctatcccataaagccgggaaggcacaacgggtggagctggcggccgccgggggcaggcacttttgagctaaggaggaagcgggggagggaaggagggcgggaaggacgcgccacctacttcctgaatcccctgcaaaccccagaggagctcggcctgcgctgcgccacgatgtccggggagtcagccaggagcttggggaagggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggtgccatccttggtaggaagctttattagaagccaaaataaagaagactatgctggcctaaaagaagaatttcgtaaagaatttaccaagctagaggaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 2 - DNA, 1593 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagtt

SEQ ID NO: 3 - DNA, 809 bp, Homo sapiens
gtaaggtgaaggtgcccagcaggctgaggcttgtgtgcaacccagaagagagctcgctaacgccagcaagaaggttcagaacagcctggctttggaaaggaatttcatcctgcccacacactgcatagcaggagggaagcatgtctactttatccaatttcacacagacgctggaagacgtcttccgaaggatttttattacttatatggacaattggcgccagaacacaacagctgagcaagaggccctccaagccaaagttgatgctgagaacttctactatgtcatcctgtacctcatggtgatgattggaatgttctctttcatcatcgtggccatcctggtgagcactgtgaaatccaagagacgggaacactccaatgacccctaccaccagtacattgtagaggactggcaggaaaagtacaagagccaaatcttgaatctagaagaatcgaaggccaccatccatgagaacattggtgcggctgggttcaaaatgtccccctgataagggagaaaggcaccaagctaacatctgacgtccagacatgaagagatgccagtgccacgaggcaaatccaaattgtctttgcttagaagaaagtgagttccttgctctctgttgagaattttcatggagattatgtggttggccaataaagatagatgacatttcaatctcagtgatttatgcttgcttgttggagcaatattttgtgctgaagacctcttttactttccgggcaagtgaatgtcattttaatcaatatcaatgatgaaaataaagccaaatttgaagaaaaaa

SEQ ID NO: 4 - DNA, 1922 bp, Homo sapiens
ctgtgagatcaccccgtgctcccagcgctcacgtcggtcctcggaaagccggggtcctccctgccttttccagcaacggtggggtggggaggcaggaagaaagcgccaacctaggaccccggagatttgcagcaaaggaagaagcgggagacgggcacttgtctgtgtctccagcgcgttcctgccccccgccgacccggcccatttctatacaaggtcgctctgcccggtctccacctcccacgtgcaggccgcggaggggctcattcccgggccctgatctcagaggcccggaatgtggctgataaatcagagactagacctgcatggcaggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggtgagagcggagggcagctcagggggattggacagcagcaatgaaagggtcctcacctgctgtcccaagaggccctcatctttcctttggaattagtgataaaggaatcagaaaatggagagactgggtgccctgaccctgtacccaaggcagtcggttcacttgggtgccatgaagggctggtgagccaggggtgggtccctgaggcttggacgcccccattcattgcaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaaggtaagcaccccctgccaccccggccgccttcccccattccagtgtgtgacactgttagagtcactttggggtttgttgtctctgggaaccacactctttgagaaaaggtcacctggacatcgcttcctcttgttaacagccttcagggccaaggggtgcctttgtggaattagtaaatgtgggcttatttcattaccatgcccacaataccttctccccacctcctacttcttatcaaaggggcagaatctcctttgggggtctgtttatcatttggcagccccccagtggtgcagaaagagaaccaaacatttcctcctggtttcctctaaactgtctatagtctcaaaggcagagagcaggatcaccagagcaatgataatccccaatttacagatgaggaaactgaggctcagagagttgcattaagcctcaaacgtctgatgactaacagggtggtgggtggcacacgatgaggtaagctcagcccctgcctccatctcccaccctaaccatcatcaccctctctctttccctgacagtgctgaggcggcattaagaggaagtcctggctgcagacacctgcttctgattccacaaggggctttttcctcaaccctgtggccgcctttgaagtgactcattttttttaatgtatttatgtatttatttgattgttttatataagatggtttcttacctttgagcacaaaatttccacggtgaaataaagtcaacattataagctttatcttttgaaactgatttgtcttggcgcattaaaaataatccctcatttcaaagaa

SEQ ID NO: 5 - DNA, 1070 bp, Homo sapiens
aaggatgactcctagacctgtctttgtcccatacttgggaagataagctgttagtttacattgccaagatgaaattcaactgatctgttttagagtaaagatcttgcgggccaacaaggaatttccttggggaggtgtgtggcctttgatctttgtagctatttagcaacaaaatgggaggcagtgttgcctgactcagtttccaagcttgactcttccctttggcatagtgagtttggagtcaagagatttttattttcccttcacaagctgaacaggagcctttggagagaacgctctggaccggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggtgccatccttggtaggaagctttattagaagccaaaataaagaagactatgctggcctaaaagaagaatttcgtaaagaatttaccaagctagaggaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 6 - DNA, 918 bp, Homo sapiens
cccttggccagcgagatgctttgacacagccccttaagatgttttaagggctatcccataaagccgggaaggcacaacgggtggagctggcggccgccgggggcaggcacttttgagctaaggaggaagcgggggagggaaggagggcgggaaggacgcgccacctacttcctgaatcccctgcaaaccccagaggagctcggcctgcgctgcgccacgatgtccggggagtcagccaggagcttggggaagggaagcgcgcccccggggccggtcccggagggctcgatccgcatctacagcatgaggttctgcccgtttgctgagaggacgcgtctagtcctgaaggccaagggaatcaggcatgaagtcatcaatatcaacctgaaaaataagcctgagtggttctttaagaaaaatccctttggtctggtgccagttctggaaaacagtcagggtcagctgatctacgagtctgccatcacctgtgagtacctggatgaagcatacccagggaagaagctgttgccggatgacccctatgagaaagcttgccagaagatgatcttagagttgttttctaaggttctgactaataagaagacgaccttctttggtggcaattctatctctatgattgattacctcatctggccctggtttgaacggctggaagcaatgaagttaaatgagtgtgtagaccacactccaaaactgaaactgtggatggcagccatgaaggaagatcccacagtctcagccctgcttactagtgagaaagactggcaaggtttcctagagctctacttacagaacagccctgaggcctgtgactatgggctctgaagggggcaggagtcagcaataaagctatgtctgatattttccttcactaatatgaataaaaaaaaaaaaaaa

SEQ ID NO: 7 - DNA, 918 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 8 - DNA, 1035 bp, Homo sapiens
gcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcggcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggagatgttacagcccagatagctcttcagcctgcactgaagttcaatggtggtggtcatatcaatcatagcattttctggacaaacctcagccctaacggtggtggagaacccaaaggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgatcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaaaaaaaaaaaa

SEQ ID NO: 9 - DNA, 708 bp, Homo sapiens
ccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaagaggaagtcctggctgcagacacctgcttctgattccacaaggggctttttcctcaaccctgtggccgcctttgaagtgactcatttttttaatgtatttatgtatttatttgattgttttatataagatggtttcttacctttgagcacaaaatttccacggtgaaataaagtcaacattataagctttaaaaaaaaaaa

SEQ ID NO: 10 - protein, 213 aa, Homo sapiens
MRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 11 - protein, 241 aa, Homo sapiens
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVPSLVGSFIRSQNKEDYAGLKEEFRKEFTKLEEVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 12 - protein, 208 aa, Homo sapiens
MSGESARSLGKGSAPPGPVPEGSIRIYSMRFCPFAERTRLVLKAKGIRHEVININLKNKPEWFFKKNPFGLVPVLENSQGQLIYESAITCEYLDEAYPGKKLLPDDPYEKACQKMILELFSKVLTNKKTTFFGGNSISMIDYLIWPWFERLEAMKLNECVDHTPKLKLWMAAMKEDPTVSALLTSEKDWQGFLELYLQNSPEACDYGL

SEQ ID NO: 13 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 14 - protein, 183 aa, Homo sapiens
MLSRAVCGTSRQLAPVLGYLGSRQKHSLPDLPYDYGALEPHINAQIMQLHHSKHHAAYVNNLNVTEEKYQEALAKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 15 - protein, 123 aa, Homo sapiens
MSTLSNFTQTLEDVFRRIFITYMDNWRQNTTAEQEALQAKVDAENFYYVILYLMVMIGMFSFIIVAILVSTVKSKRREHSNDPYHQYIVEDWQEKYKSQILNLEESKATIHENIGAAGFKMSP

SEQ ID NO: 16 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

(19) United States
(12) Patent Application Publication
(10) Pub. No.: US 2013/0338027 A1
(43) Pub. Date: Dec. 19, 2013

(54) Predictive Markers For Cancer and Metabolic Syndrome

(71) Applicant: Nuclea Biotechnologies, Inc., Pittsfield, MA (US)

(72) Inventor: Patrick J. Muraca, Pittsfield, MA (US)

(21) Appl. No.: 13/916,635

(22) Filed: Jun. 13, 2013

Related U.S. Application Data
(60) Provisional application No. 61/660,018, filed on Jun. 15, 2012.
(60) Provisional application No. 61/727,323, filed on Nov. 16, 2012.
(60) Provisional application No. 61/766,931, filed on Feb. 20, 2013.

(51) Int. Cl. G01N 33/68; C12Q 1/68

(52) U.S. Cl. CPC G01N 33/6893; C12Q 1/6886
USPC 506/9; 435/6.11; 435/7.1; 530/389.7; 435/7.92

(57) ABSTRACT

Disclosed are predictive biomarkers and methods of use for the determination of insulin resistance and sensitivity, in addition to cardiovascular disease and risk associated with obesity. Methods for the stratification of patients along continuum of susceptibility to cardiometabolic risk, including prediction and progression to metabolic syndrome are also provided.

### CROSS REFERENCE TO RELATED APPLICATIONS

This application claims priority to U.S. Provisional Application No. 61/660,018, filed Jun. 15, 2012, U.S. Provisional Application No. 61/727,323, filed Nov. 16, 2012, and U.S. Provisional Application No. 61/766,931, filed Feb. 20, 2013, the content of each of which is incorporated by reference in their entirety.

### REFERENCE TO SEQUENCE LISTING

The present application is being filed along with a Sequence Listing in electronic format. The Sequence Listing file, entitled 20151018USSEQLST.txt, was created on Jun. 13, 2013 and is 26,131 bytes in size. The information in electronic format of the Sequence Listing is incorporated herein by reference in its entirety.

### FIELD OF THE INVENTION

The present invention provides predictive biomarkers and methods of use for the determination of insulin resistance and sensitivity, in addition to cardiovascular disease and risk associated with obesity. Methods for the stratification of patients along continuum of susceptibility to cardiometabolic risk are also provided.

### BACKGROUND OF THE INVENTION

As obesity becomes more prevalent, a fundamental diagnostic challenge is to identify among the obese population individuals whose obesity causes more metabolic stress and who, consequently, are more likely to develop type 2 diabetes and cardiovascular diseases often leading to metabolic syndrome (MS) and/or congestive heart failure (CHF). Thus, early identification of increased susceptibility to cardiometabolic risk carries enormous therapeutic and economic implications. Whereas there is general agreement that, at least from an epidemiologic perspective, obesity poses a cardiometabolic risk, it is also true that not all obese individuals develop cardiometabolic complications. Conversely, it is also true that some individuals develop metabolic complications inappropriately severe for their degree of obesity. This suggests that the fat mass per se may not be the best predictor of the propensity to progress (or not) to more severe health conditions. It also suggests that a marker that could indicate the metabolic stress posed by a specific degree of obesity may be particularly useful.

Accumulating evidence suggests that lipid metabolism is as important, if not more important, to diabetes as is carbohydrate metabolism. The anabolic effects of insulin are not limited to facilitating glucose uptake. In fact, insulin is the most lipogenic hormone and exerts important effects on protein metabolism. Furthermore, insulin is an important regulator of fatty acid synthase (FASN), a key enzyme in de novo lipogenesis. Coordinately, acetyl CoA carboxylase (ACC) along with FASN determines the lipogenic flux from malonyl CoA into palmitate. Expression of the FASN gene is primarily regulated by hormonal and nutritional signals, and insulin particularly not only increases the rate of FASN gene transcription in murine cell lines and primary human adipocytes but also increases human FASN gene expression and FASN enzymatic activity. Conversely, FASN is markedly inactivated under conditions of insulin resistance.

Polymorphisms in the FASN gene have also been investigated in connection with tumor expression, body mass index (BMI), prostate cancer risk and survival (Nguyen, et al, J. Clin. Oncol. 28: 2010, 3958-3964) indicating a potential overlap between cancer and cardiometabolic risk. In these studies, four of five SNPs in the FASN gene were associated with lethal prostate cancer. Further, it was suggested that interaction of body mass index (BMI) with FAS polymorphisms and FASN tumor expression linked obesity and poor prostate cancer outcome, especially in overweight men. However, the studies were limited to SNPs in the FASN gene where the patient population was limited to Caucasian subjects.

It is therefore hypothesized that a marker related to the de novo lipogenic pathway, and more specifically FASN, or FASN in combination with other biomarkers, might provide some insights on the level of impairment of insulin sensitivity and metabolic stress. As such, the levels of FASN, including variant forms of FASN, such as single nucleotide polymorphisms may serve as a biomarker for metabolic syndrome or any of the component conditions associated with the syndrome.

In support of the foregoing hypothesis, that FASN is a potential marker for metabolic stress, there is evidence that FASN, an intracellular protein, can also be detected at increased levels in the extracellular space of human cancer cells. It has been suggested that when FASN is excessively accumulated beyond the metabolic needs of the cellular system, it is exported to the extracellular space of the cell.

### SUMMARY OF THE INVENTION

The present invention provides predictive biomarkers and methods of use for the determination of insulin resistance and sensitivity, in addition to cardiovascular disease, diabetes and certain cancers. Methods for the stratification of patients along continuum of susceptibility to cardiometabolic risk and/or metabolic syndrome are also provided.

The present invention provides a method for predicting the incidence of metabolic syndrome in a subject comprising determining the level of one or more FASN SNPs such as, but not limited to, rs4246444, rs6502051 and rs12949488 in a sample obtained from a subject and stratifying the subject as likely to develop metabolic syndrome based on the level of one or more FASN SNPs. The level of the one or more FASN SNPs may be greater than a 0.7 signal to noise ratio. The subject may have been diagnosed with type 2 diabetes and may also be resistant to insulin.

The present invention provides a method for predicting the incidence of metabolic syndrome in a subject comprising determining the level of FASN or FASN in combination with USP2A in a sample obtained from the subject and stratifying the subject as likely to develop metabolic syndrome based on the level of FASN or FASN in combination with USP2A where the detection rate of either FASN or FASN in combination with USP2A may be independently 0.90 or greater. The subject may have been diagnosed with type 2 diabetes and may also be resistant to insulin. The protein expression levels may be measured by methods such as, but not limited to, immunohistochemical assay which may utilize one or more FASN specific antibodies. The FASN specific antibodies may further contain a detectable label.

The methods of predicting the incidence of metabolic syndrome may further comprise measuring one or more clinical management parameter such as, but not limited to, blood pressure, body mass index (BMI), levels of insulin, blood sugar, triglycerides, HDL, LDL and C-reactive protein.

The present invention provides an immunohistochemical kit or assay to predict or detect metabolic syndrome comprising one or more FASN specific antibodies which may comprise a detectable label. The kit or assay may further comprise a probe targeting the USP2a gene or protein.

The present invention provides a method for predicting the recurrence or aggressiveness of prostate cancer in a subject comprising determining the level of one or more SNPs such as, but not limited to, rs1447295, rs6983267, rs10993994, rs7127900, rs12621278, rs170021918, rs10486567, rs1512268 and rs12949488 in a sample obtained from the subject and stratifying the subject as likely to have a recurrence of cancer or an aggressive form of cancer based on the level of one or more SNPs. The level of the one or more SNPs may be elevated over the background.

The present invention provides a method for predicting the recurrence or aggressiveness of prostate cancer in a subject comprising determining the level of at least one SNP or expression product of one or more genes, the genes such as, but not limited to, FTO (fat mass and obesity associated) gene, MC4R (melanocortin 4 receptor), TMEM18 (transmembrane protein 18), GNPDA2 (glucosamine-6-phosphate deaminase 2), ETV5 (Ets variant 5), BDNF (brain derived neurotrophic factor), SH2B1 (SH2B adapter protein 1), and PCSK1 (proprotein convertase subtilisin/kexin type 1) in a sample obtained from the subject and stratifying the subject as likely to have a recurrence of cancer or an aggressive form of cancer based on the level of at least one SNP or expression product. The level of the at least one SNP or expression product may be elevated over the background. The expression product may be a protein and the levels of the protein may be measured by immunohistochemical assay which may utilize one or more antibodies.

The present invention provides a method of predicting whether a subject afflicted with early stage heart failure will progress to a later stage comprising obtaining a biologic sample from the subject, determining the level of one or more biomarkers selected from the group consisting of FASN, FASN SNPs and USP2A and stratifying the subject as likely to progress to a later stage of heart failure based on the level of said one or more biomarker. This method may also be applied to a subject further afflicted with metabolic syndrome. In another embodiment, this method may also be applied to a subject diagnosed with type 2 diabetes. In another embodiment, this method may be applied to a subject that is insulin resistant.

The present invention provides a method of predicting whether a subject afflicted with metabolic syndrome will develop heart failure comprising obtaining a biologic sample from the subject, determining the level of one or more biomarkers selected from the group consisting of FASN, USP2A, GSTΩ1, SOD2, KCNE2 and BNP in said biologic sample and stratifying the subjects as likely to develop heart failure based on the expression level of said one or more biomarkers. In a further embodiment, the subject has also been diagnosed with type 2 diabetes. In a further embodiment, the subject is also insulin resistant. In another embodiment, protein expression levels are measured.

The invention provides a method of predicting the incidence of metabolic syndrome in a subject comprising obtaining a biologic sample from the subject and determining the expression level of one or more biomarkers in the biologic sample wherein the biomarkers are selected from the group consisting of FASN, USP2A, GSTΩ1, SOD2, KCNE2 and BNP. In a further embodiment, the biologic sample obtained is selected from the group consisting of blood, peripheral blood mononuclear cells (PBMC), isolated blood cells, serum and plasma. In another embodiment, protein expression levels are measured by immunoassay. In a further embodiment, the immunoassay is an enzyme-linked immunosorbent assay (ELISA). In another embodiment, the expression level of two biomarkers is determined.

### DETAILED DESCRIPTION OF THE INVENTION

The invention relates to compositions, methods and assays for detecting, screening for, or diagnosing conditions including, but not limited to metabolic syndrome, heart failure, insulin resistance and/or insulin sensitivity; staging or stratifying subjects; and determining the progression of, regression of and/or survival from metabolic syndrome.

As used herein, the term “metabolic syndrome” or “MS” refers to a group of risk factors that occur together and increase an individual's risk for coronary artery disease, heart failure (HF) [also referred to herein as congestive heart failure (CHF)], stroke, and Type 2 diabetes. Metabolic syndrome in Type 2 diabetes is characterized by the presentation of hyperinsulinemia, meaning a fasting insulin in the upper 25% of the diabetic population, e.g., elevated fasting blood glucose.

According to the International Diabetes Foundation (IDF), metabolic syndrome presents with central obesity and any two of the following: (1) raised triglycerides (TG) of >150 mg/dL (1.7 mmol/L), or specific treatment for increased triglycerides; (2) reduced high density lipoproteins (HDL) of <40 mg/dL (1.03 mmol/L) in males <50 mg/dL (1.29 mmol/L in females; (3) raised blood pressure (BP) with systolic >130 or diastolic >85 mm Hg or treatment for hypertension and (4) raised fasting plasma glucose (FPG)>100 mg/dL (5.6 mmol/L) or previous diagnosis of type 2 diabetes.

Metabolic syndrome may also be defined as presentation of hyperinsulinemia and any two of the following: (1) abdominal obesity (waist/hip ration >0.90 or BMI 30 kg/m²), (2) dyslipidemia (triglycerides (TG)>1.7 or high density lipoprotein (HDL)<0.9 mmol/L) and (3) hypertension (blood pressure (BP)>140/90 or use of antihypertensive medication).

Even with these defined metrics there are still no early predictive markers of the metabolic risk posed by obesity or fat mass status. Thus, new therapeutic targets and biomarkers useful for diagnostic and preventive purposes, ideally pathogenically linked, are urgently needed.

### Metabolic Syndrome and Congestive Heart Failure

Markers indicative of MS have been shown to be predictive of heart failure (HF), also known as congestive heart failure (CHF) (Suzuki, T. et al., Metabolic Syndrome, Inflammation, and the incident heart failure in the elderly: the cardiovascular health study. Circ Heart Fail. 2008 November; 1(4):242-8). CHF, generally is defined as the inability of the heart to supply sufficient blood flow to meet the body's needs. It has various diagnostic criteria, and the term heart failure is often incorrectly used to describe other cardiac-related illnesses, such as myocardial infarction (heart attack) or cardiac arrest.

Common causes of heart failure include cardiovascular complications associated with MS including, but not limited to myocardial infarction (heart attacks) and other forms of ischemic heart disease, hypertension, valvular heart disease, and cardiomyopathy. McMurray J J, Pfeffer M A (2005), “Heart failure”, Lancet 365 (9474): 1877-89. Heart failure can cause a number of symptoms including shortness of breath (typically worse when lying flat, which is called orthopnea), coughing, chronic venous congestion, ankle swelling, and exercise intolerance. Heart failure is often undiagnosed because of a lack of a universally agreed definition and challenges in definitive diagnosis. Treatment commonly consists of lifestyle measures (such as smoking cessation, light exercise including breathing protocols, decreased salt intake and other dietary changes) and medications, and sometimes devices (pacemaker) or even surgery.

Functional classification for the stages of heart failure generally relies on the New York Heart Association Functional Classification. Criteria Committee, New York Heart Association. Diseases of the heart and blood vessels. Nomenclature and criteria for diagnosis, 6th ed. Boston: Little, Brown and co, 1964; 114. The classes (1-IV) are:

Class I: no limitation is experienced in any activities; there are no symptoms from ordinary activities.

Class II: slight, mild limitation of activity; the patient is comfortable at rest or with mild exertion.

Class III: marked limitation of any activity; the patient is comfortable only at rest.

Class IV: any physical activity brings on discomfort and symptoms occur at rest.

The NYHA score documents severity of symptoms, and can be used to assess response to treatment. While its use is widespread, the NYHA score is not very reproducible and doesn't reliably predict the walking distance or exercise tolerance on formal stress testing. Raphael C, Briscoe C, Davies J, et al. (2007) “Limitations of the New York Heart Association functional classification system and self-reported walking distances in chronic heart failure”, Heart 93(4): 476-82.

In its 2001 guidelines the American College of Cardiology/American Heart Association working group introduced an alternate classification/staging system describing four stages of heart failure:

Stage A: At high risk for developing HF in the future but without structural heart disease or symptoms of HF;

Stage B: Structural heart disease but without signs or sympoms of HF;

Stage C: Structural heart disease but with prior or current symptoms of HF;

Stage D: Refractory HF requiring specialized interventions (hospital-based support, a heart transplant or palliative care).

Hunt S A, Abraham W T, Chin M H, et al. (2005) “ACC/AHA 2005 Guideline Update for the Diagnosis and Management of Chronic Heart Failure in the Adult”, Circulation, 112 (12): e154-235; Jessup M, et al., “2009 Focused Update: ACCF/AHA Guidelines for the Diagnosis and Management of Heart Failure in Adults”, Circulation (2009), 119:1977-2016. The ACC staging system is useful in that Stage A encompasses “pre-heart failure”, a stage at which therapeutic intervention can presumably prevent progression to overt symptoms. ACC stage A does not have a corresponding NYHA class. ACC Stage B would correspond to NYHA Class I. ACC Stage C corresponds to NYHA Classes II and III, while ACC Stage D overlaps with NYHA Class IV.

No system of diagnostic criteria has been agreed upon as the gold standard for heart failure. Commonly used systems are the “Framingham criteria” (derived from the Framingham Heart Study), McKee P A, Castelli W P, McNamara P M, Kannel W B (1971) “The natural history of congestive heart failure: the Framingham study”, N. Engl. J. Med. 285 (26): 1441-6; the “Boston criteria”, Carlson K J, Lee D C, Goroll A H, Leahy M, Johnson R A (1985) “An analysis of physicians' reasons for prescribing long-term digitalis therapy in outpatients” Journal of chronic diseases 38 (9): 733-9; the “Duke criteria”, Harlan W R, Oberman A, Grimm R, Rosati R A (1977) “Chronic congestive heart failure in coronary artery disease: clinical criteria”, Ann. Intern. Med. 86 (2): 133-8; and (in the setting of acute myocardial infarction) the “Killip class” Killip T, Kimball J T (1967) “Treatment of myocardial infarction in a coronary care unit: A two year experience with 250 patients”, Am. J. Cardiol. 20 (4): 457-64.

Other methods used to aid in diagnosing HF include imaging (e.g., echocardiography), chest X-rays, electrophysiology (e.g., an electrocardiogram (ECG/EKG) may be used to identify arrhythmias, ischemic heart disease, right and left ventricular hypertrophy, and presence of conduction delay or abnormalities); and blood tests.

Blood tests routinely performed include electrolytes (sodium, potassium), measures of renal function, liver function tests, thyroid function tests, a complete blood count, and often C-reactive protein (also a diagnostic marker for MS) if infection is suspected. An elevated B-type natriuretic peptide (BNP) is a specific test indicative of heart failure. Additionally, BNP can be used to differentiate between causes of dyspnea due to heart failure from other causes of dyspnea. If myocardial infarction is suspected, various cardiac markers may be used. BNP is a useful indicator for heart failure and left ventricular systolic dysfunction. Ewald B, Ewald D, Thakkinstian A, Attia J (2008), “Meta-analysis of B type natriuretic peptide and N-terminal pro B natriuretic peptide in the diagnosis of clinical heart failure and population screening for left ventricular systolic dysfunction”, Intern Med J, 38 (2):101-13.

Prognosis in heart failure can be assessed in multiple ways including clinical prediction rules and cardiopulmonary exercise testing. Clinical prediction rules use a composite of clinical factors such as lab tests and blood pressure to estimate prognosis. Among several clinical prediction rules for prognosing acute heart failure, the ‘EFFECT rule’ slightly outperformed other rules in stratifying patients and identifying those at low risk of death during hospitalization or within 30 days. Auble T E, Hsieh M, McCausland J B, Yealy D M (2007) “Comparison of four clinical prediction rules for estimating risk in heart failure”, Annals of emergency medicine, 50(2): 127-35, 135.e1-2.

Biomarkers that are predictive of the risk of a HF patient advancing to a later stage of disease, herein referred to as “HF-related biomarkers,” include, but are not limited to glutathione S-transferase omega-1 (GSTΩ1), superoxide dismutase 2 (SOD2), potassium voltage-gated channel subfamily E member 2 (KCNE2), fatty acid synthase (FASN) and B-type natriuretic peptide (BNP). Both GSTΩ1 and SOD2 are involved in oxidative stress management while BNP and KCNE2 proteins modulate blood pressure and cardiac contraction respectively. Changes in levels of oxidative stress and cardiovascular function are also components of MS (Hutcheson, R. et al., The metabolic syndrome, oidative stress, environment, and cardiovascular disease: the great exploration. Exp Diabetes Res. 2012; 2012:271028) indicating a link between MS and HF and highlighting the importance of these biomarkers in the diagnostic evaluation of these diseases. Further, it has been shown that the fatty acid synthase gene (FASN), a biomarker for MS, is active in cardiomyocytes after heart failure. Razani et al (Razani, B. et al., Fatty acid synthase modulates homeostatic responses to myocardial stress. J Biol Chem. 2011 Sep. 2; 286(35):30949-61) demonstrate increases in FASN in two separate mouse models of heart failure as well as in human hearts with end stage cardiomyopathy. The authors suggest that in a weakened state, the heart may turn to endogenous lipids, such as the ones produced by FASN, to maintain cardiomyocyte homeostasis. This study demonstrates the ability of FASN to act as a biomarker for heart failure. In some embodiments, these biomarkers may be used to determine risk of developing MS and/or HF. In some embodiments, these biomarkers may be used to diagnose MS and/or HF that the subject is experiening or has experienced.

In some embodiments of the present invention, combinations of HF-biomarkers may be detected in a subject to diagnose, prognose or otherwise evaluate an individual with MS. In some embodiments combinations of HF-related biomarkers may be detected in an individual with MS to determine the risk of HF. In some embodiments, HF biomarkers biomarkers may be evaluated along with other biomarkers of MS to enhance the diagnostic and/or prognostic values of these biomarkers analyzed separately.

The present invention provides compositions, methods, kits and other clinical tools to augment traditional diagnostic, prognostic and/or therapeutic paradigms. Combination approaches using one or more biomarkers in the determination of the value of one or more clinical management parameters also are envisioned. For example methods of this invention that measure FASN and USP2A biomarkers, alone or in combination can provide potentially superior results to diagnostic assays measuring just one of these biomarkers. A dual or multi-biomarker approach would provide even further superiority. Any dual, or multiple, biomarker approach (with or without companion testing such as blood pressure, triglycerides, etc) thus reduces the number of patients that are predicted not to benefit from treatment, and thus potentially reduces the number of patients that fail to receive treatment that may extend or improve their life significantly.

### Clinical Management Parameters

Clinical management parameters addressed by the present invention include, but are not limited to, survival in years, disease related death, early or late response to insulin and resistance, degree of regression, responsiveness to treatment, effectiveness of treatment, the likelihood of progression of a condition on to a more severe disease such as one or more cancers, blood pressure, body mass index (BMI), levels of insulin, blood sugar, triglycerides, HDL, LDL, C-reactive protein, as well as biomarker status such as levels of FASN, USP2A, GSTΩ1, SOD2, KCNE2, BNP or other genes or a SNP of FASN, GSTΩ1, SOD2, KCNE2, BNP or USP2A, or any metabolic related gene.

In one embodiment, the present invention provides certain predictor variables which are single nucleotide polymorphisms (SNPs) of select genes. These genes include fatty acid synthase (FASN), ubiquitin specific protease 2 (USP2a), glutathione-S-transferase omega-1 (GSTΩ1), superoxide dismutase 2 (SOD2), potassium voltage-gated channel subfamily E member 2 (KCNE2), B-type natriuretic peptide (BNP) and other metabolic pathway genes.

Advantageously, practice of the present invention can result in reduced harms caused by screening (resulting in false positives or false negative) and unnecessary subsequent evaluations and therapy.

In some embodiments, the invention relates to compositions, methods and assays for detecting, screening for, or diagnosing heart failure (HF); staging or stratifying HF patients; and determining the progression of, regression of and/or survival from HF.

In doing so, the present invention provides methods, algorithms and other clinical tools to augment traditional diagnostic, prognostic and/or therapeutic paradigms. Combination approaches using one or more biomarkers in the determination of the value of one or more clinical management parameters also are envisioned. For example methods of this invention that measure FASN, USP2a, GSTΩ1, SOD2, KCNE2 and BNP can provide potentially superior results to diagnostic assays measuring just one of these biomarkers, as illustrated by the data presented herein. This dual biomarker approach, in combination with imaging techniques would provide even further superiority. Any dual, or multiple, biomarker approach (with or without companion imaging) thus reduces the number of patients that are predicted not to benefit from treatment, and thus potentially reduces the number of patients that fail to receive treatment that may extend their lives significantly.

Clinical management parameters addressed by the present invention include survival in years, disease related death, degree of progression, responsiveness to treatment and effectiveness of treatment (e.g., increased cardiac output).

Having found that expression of two or more of GSTΩ1, SOD2, KCNE2 and BNP is a superior predictor of many of the clinical management parameters important to clinicians treating patients having or suspected of having HF, the present invention involves the rapid and accurate identification of these markers in cells and/or serum. Given the relationship between the gene function of these genes and aspects of MS, the present invention also contemplates the use of the information obtained from biomarker analysis of these genes to predict the incidence of MS in a subject.

In one embodiment, a method generally comprises the following steps: (a) obtaining a biological sample (optimally containing cells or other cell or fluid) from a subject; (b) contacting the sample with a detection agent specific for one of the following biomarker sets: FASN; FASN variant, isoforms or SNP; one or more of GSTΩ1, SOD2, KCNE2 and BNP or one or more of FASN, USP2a, GSTΩ1, SOD2, KCNE2 and BNP; (c) detecting the presence, amount or levels of the biomarker sets in (b); and (d) correlating the presence, amount or levels of the biomarkers determined with the one or more clinical management parameters in order to aid in the prevention, diagnosis or treatment of a disease or condition such as metabolic syndrome.

The biological sample may be cells, and preferably is serum or plasma containing cells. However, the cells also may be obtained from tissue samples or cell cultures such as in ex vivo or in situ methods.

The detection agent may be a nucleic acid probe SNP, protein specific for FASN, or an anti-FASN antibody.

In one embodiment, a method generally comprises the following steps: (a) obtaining a biological sample (optimally containing cells or other cell or fluid) from a subject; (b) contacting the sample with a detection agent specific for one of the following marker sets: GSTΩ1 and SOD2; KCNE2 and BNP; GSTΩ1 and SOD2, together with one of KCNE2 or BNP; KCNE2 and BNP, together with one of GSTΩ1 and SOD2; GSTΩ1, SOD2, KCNE2 and BNP; FASN and/or USP2a with one or more of SOD2; GSTΩ1, SOD2, KCNE2 and BNP; (c) detecting the presence, amount or levels of the markers in (b); and (d) correlating the presence, amount or levels of the markers in order to aid in the prevention, diagnosis or treatment of a condition such as heart failure (HF). Step (d) may further include correlating the marker levels with one or more clinical management parameters and/or imaging data. Clinical management parameters may include, for example, stress testing, cardiac echocardiogram, and cardiac enzymes in blood.

The biological sample may be cells or blood, and preferably is serum or plasma containing cells [including, for example and without limitation peripheral blood mononuclear cells (PBMCs)]. However, the cells also may be obtained from cell cultures such as in ex vivo or in situ methods.

The detection agent may a nucleic acid probe specific for one or more of FASN, USP2a, GSTΩ1, SOD2, KCNE2 and BNP, or an antibody specific for one or more of FASN, USP2a, GSTΩ1, SOD2, KCNE2 and BNP.

### FASN Probes

The present invention also provides nucleic acid based probes useful in the detection of the FASN gene or protein in a biological sample. To this end, the present invention includes nucleic acid sequences specific for segments of a human FASN gene which are used in methods of detecting FASN-specific sequences, including SNPs, in nucleic acids prepared from a biological sample. The invention further includes nucleic acid sequences specific for segments of other genetic markers, such as a human USP2a, pAKT, NPY, and/or AMACR.

Other genes whose measurement of gene expression, protein levels or variants (including SNPs) may have diagnostic, prognostic or therapeutic value, alone or in combination include GSTΩ1 (glutathione-S-transferase omega-1), SOD2 (superoxide dismutase 2), KCNE2 (potassium voltage-gated channel subfamily E member 2), BNP (B-type natriuretic peptide), the FTO (fat mass and obesity associated) gene, MC4R (melanocortin 4 receptor), TMEM18 (transmembrane protein 18), GNPDA2 (glucosamine-6-phosphate deaminase 2; variants of which are associated with obesity), ETV5 (Ets variant 5), BDNF (brain derived neurotrophic factor), SH2B 1 (SH2B adapter protein 1), PCSK1 (proprotein convertase subtilisin/kexin type 1; which regulates insulin biosynthesis), and ATM (ataxia telangiectasia mutated). Any of the genes or proteins may be measured in a subject who has received no treatment for metabolic syndrome or those who have received treatment such as with metformin.

The biological sample may include, for example, blood, peripheral blood mononuclear cells (PBMC), isolated blood cells, serum, plasma, lymph node, breast or breast cyst, kidney, liver, lung, muscle, stomach or intestinal tissue. The invention also includes preferred methods that combine nucleic acid sequences for amplifying and detecting FASN-specific sequences, including SNPs, USP2a, pAKT, NPY, GSTΩ1, SOD2, KCNE2, BNP and/or AMACR sequences, individually or in combination.

Preferred probes, primers and promoter-primers of the present invention are used for detecting and quantifying the FASN-specific RNA species including variants, isoforms or SNPs. Other embodiments of the invention include methods for detecting USP2a, pAKT, GSTΩ1, SOD2, KCNE2, BNP, NPY, and/or AMACR RNA species, individually or in combination with each other or FASN sequences. Moreover, detection of these markers individually and in combination, are clinically important because cells from individual patients may express one or more of the markers, such that detecting one or more of the markers decreases the potential of false negatives during diagnosis that might otherwise result if the presence of only one marker was tested.

In one embodiment, commercial antibodies may be used to detect expression. One such antibody for USP2a is the USP2 Antibody (N-term) from Abgent (San Diego, Calif.; Cat. #AP2131a).

### FASN Antibodies

In one embodiment, the present invention utilizes anti-FASN antibodies and ELISA assay. The anti-FASN antibodies preferably are those disclosed in PCT Publication PCT/US2010/030545 published Oct. 14, 2010, and PCT/US2010/046773 published Mar. 17, 2011, respectively. The anti-FASN antibodies may also be BD antibodies (BD Transduction Laboratories, Franklin Lakes N.J.) such as, but not limited to, fatty acid synthase (Catalog No. 610963).

The antibodies used in the present invention for detection or capture of FASN are novel anti-FASN antibodies that are highly specific for human FASN.

In one embodiment, commercial antibodies for the detection of FASN are used. For IHC the antibodies which may be used are the human anti-FASN Antibody, Affinity Purified (Catalog No. A301-324A) from Bethyl Laboratories (Montgomery, Tex.) and for ELISA studies, antibodies which may be used include the Fatty Acid Synthase Antibody Pair (Catalog No. H00002194-AP11) from Novus Biologicals (Littleton, Colo.). The pair contains a Capture antibody which is rabbit affinity purified polyclonal anti-FASN (100 ug) and a Detection antibody which is mouse monoclonal anti-FASN, IgG1 Kappa (20 ug).

### In Situ Hybridization (ISH) and Fluorescence In Situ Hybridization (FISH)

The present invention provides methods of detecting target nucleic acids via in situ hybridization and fluorescent in situ hybridization using novel probes. The methods of in situ hybridization were first developed in 1969 and many improvements have been made since. The basic technique utilizes hybridization kinetics for RNA and/or DNA via hydrogen bonding. By labeling sequences of DNA or RNA of sufficient length (approximately 50-300 base pairs), selective probes can be made to detect particular sequences of DNA or RNA. The application of these probes to tissue sections allows DNA or RNA to be localized within tissue regions and cell types. Methods of probe design are known to those of skill in the art. Detection of hybridized probe and target may be performed in several ways known in the art. Most prominently is through the use of detection labels (also referred to herein as “detectable labels”) attached to the probes. Probes of the present invention may be single or double stranded and may be DNA, RNA, or mixtures of DNA and RNA. They may also constitute any nucleic acid based construct. Detectable labels for the probes of the present invention may be radioactive or non-radioactive and the design and use of such labels is well known in the art.

### Gene Expression and Localization of Expression

In one embodiment of the invention, FASN expression is measured relative to the expression of one or more additional genes and/or at one or more different biopsy sites or at a different blood or serum draw time. Comparisons of gene expression within the site and/or at a different time allow conclusions to be drawn about the status of a site or the subject and whether a condition such as (but not limited to) any of the features of metabolic syndrome require further monitoring or clinical management. These conclusions then allow for improved predictions, such as (but not limited to) progression of insulin sensitivity and resistance and overall health outcomes. One set of genes which are particularly useful in these methods includes FASN combined with one or more of USP2a, GSTΩ1, SOD2, KCNE2, BNP, pAKT and NPY. Additional patient parameters may also be combined with the gene expression data to improve the predictive power of the method. Such patient parameters include age, gender, race, BMI, weight, height or other clinical management parameter.

### FASN and Insulin Resistance

In one embodiment, FASN expression levels are used as a predictor of insulin resistance. In another embodiment, the expression levels of FASN may be used in combination with USP2a, GSTΩ1, SOD2, KCNE2, BNP, NPY and/or one or more other parameters described herein to predict insulin resistance. As a non-limiting example, FASN alone or in combination with USP2a, GSTΩ1, SOD2, KCNE2, BNP, NPY and/or one or more other parameters described herein may be an accurate predictor of insulin resistance in Type 2 Diabetes.

### FASN and Insulin Sensitivity

In one embodiment, FASN expression levels are used as a predictor of insulin sensitivity.

### FASN and USP2a

In one embodiment, the present invention provides for the use of combinations of predictors or biomarkers which, heretofore, have not been known as significant collective indicator combinations. These combinations may form the basis of methods, assays or kits useful in the clinical management of metabolic syndrome and type 2 diabetes.

### Gene/Protein Expression Profiles

Also described herein are compositions and methods for employing gene (including SNPs of genes) and protein expression profiles in prognosis, prediction and management of treatment paradigms associated with metabolic syndrome.

The gene expression profiles (GEPs) and protein expression profiles (PEPs) (collectively the GPEPs) of the present invention provides the clinician with a prognostic tool capable of providing valuable information that can positively affect management of a disease such as metabolic syndrome and type 2 diabetes. According to the present invention, physicians can assay the suspect tissue for the presence of members of a GPEP, and can identify with a high degree of accuracy those patients whose condition is likely to progress, regress or become a more aggressive from of the disease such as cardiometabolic disorders. This information, taken together with other available clinical information including disease monitoring data, allows more effective management of the disease.

In one aspect of the invention, the expression of genes (including SNPs of those genes) or proteins in serum or plasma from a patient is assayed using array or immunohistochemistry techniques to identify the expression of genes or proteins in a GPEP.

Certain methods of the present invention comprise (a) obtaining a biological sample (preferably tissue or serum) (b) contacting the sample with nucleic acid probes or antibodies specific for one or more members of a GPEP, PEP or GEP and (c) determining whether one or more of the members of the profile are present or up-regulated (over-expressed).

The predictive value of the GPEPs for determining the likelihood of disease appearance or progression may increase with the number of the members found to be present or up-regulated. In one embodiment, SNPs of FASN are the single biomarker tested and identified in a sample. In another embodiment, at least about two, more preferably at least about four, and most preferably about seven, of the genes and/or proteins in a GPEP are evaluated for expression. Preferably, expression of at least one reference protein or gene is also measured at the same time and under the same conditions.

In one embodiment, the present invention comprises gene expression profiles and/or protein expression profiles that are indicative of the likelihood of presence, recurrence or progression to metabolic syndrome, heart failure or type 2 diabetes in a subject. In this embodiment, the present method comprises (a) obtaining a biological sample of a subject afflicted with or having clinical or patient parameters suggesting possible metabolic syndrome or heart failure; (b) contacting the sample with nucleic acid probes to the GEP or antibodies to the proteins of a PEP and (c) determining whether two or more of the members of the profile are present, up-regulated (over-expressed). The biological sample preferably is a sample of the subject's serum. Preferably, expression of at least one reference gene also is measured. Reference genes may include beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

When measuring SNPs, the wild type or alternatively a separate allele may be used as a control or comparison.

The present invention further comprises assays for determining the gene and/or protein expression profile in a biological sample, and instructions for using the assay. The assay may be based on detection of nucleic acids (e.g., using nucleic acid probes specific for the nucleic acids of interest) or proteins or peptides (e.g., using nucleic acid probes or antibodies specific for the proteins/peptides of interest). In one embodiment, the assay comprises an immunohistochemistry (IHC) test in which tissue samples, preferably arrayed in a tissue microarray (TMA), are contacted with antibodies specific for the proteins/peptides identified in the GPEP where detection is taken as being indicative of a relationship between the detected gene and one or more clinical management parameters.

In one embodiment, the assay comprises an inducible protein (IP) or ELISA test in which biologic samples are contacted with antibodies specific for the proteins/peptides identified in the GPEP where detection is taken as being indicative of a relationship between the detected gene and one or more clinical management parameters or patient parameters.

Inclusion of any of the biomarker or diagnostic methods described herein as part of treatment and/or monitoring regimens to predict the progression to, or effectiveness of treatment of, a subject with any therapeutic provides an advantage over treatment or monitoring regimens that do not include such a biomarker or diagnostic step, in that only that patient population which needs or derives most benefit from such therapy or monitoring need be treated or monitored, and in particular, patients who are predicted not to need or benefit from treatment (where progression is not predicted) with any therapy need not be treated.

The present invention further provides a method for treating a patient who may have metabolic syndrome or heart failure, comprising the step of diagnosing a patient's likely progression to metabolic syndrome or heart failure using one or more GPEP signatures to evaluate conditions such as (but not limited to) insulin resistance or insulin sensitivity; and a step of administering the patient an appropriate treatment regimen for any of the component conditions associated with metabolic syndrome or heart failure given the patient's age, or other therapeutically relevant criteria.

### Determination of Gene Expression Profiles

Methods used to identify gene expression profiles indicative of whether a patient's condition is likely to progress to metabolic syndrome and/or heart failure are generally described here and further described in the Examples herein. Other methods for identifying gene and/or protein expression profiles are known; any of these alternative methods also could be used. See, e.g., Chen et al., NEJM, 356(1):11-20 (2007); Lu et al., PLOS Med., 3(12):e467 (2006); Wang et al., J. Clin. Oncol., 2299):1564 (2004); Golub et al., Science, 286:531-537 (1999).

In one method, parallel testing in which, in one track, those genes are identified which are over-/under-expressed as compared to normal tissue and/or disease tissue from patients that experienced different outcomes; and, in a second track, those genes are identified comprising chromosomal insertions or deletions as compared to the same normal and disease samples. These two tracks of analysis produce two sets of data. The data are analyzed and correlated using an algorithm which identifies the genes of the gene expression profile (i.e., those genes that are differentially expressed in the tissue, serum and/or plasma of interest). Positive and negative controls may be employed to normalize the results, including eliminating those genes and proteins that also are differentially expressed in normal tissues from the same patients, and is disease tissue having a different outcome, and confirming that the gene expression profile is unique to the disorder of interest.

As an initial step, biological samples are acquired from patients presenting with either metabolic syndrome, heart failure or underlying conditions indicative of metabolic syndrome or heart failure. Tissue samples are also obtained from patients diagnosed as having progressed to, for example, metabolic syndrome, heart failure or with type 2 diabetes. Clinical information associated with each sample, including treatment with gluconeogenic, such as metformin, or gluco-inhibitory drugs, or other treatment, outcome of the treatments and recurrence or progression of the disease, is recorded in a database. Clinical information also includes information such as age, sex, medical history, treatment history, symptoms, family history, recurrence (yes/no), etc. Samples of normal tissue of different types (e.g., tissue, serum, etc) as well as samples of non-metabolic syndrome or non-diabetic can be used as controls.

Gene expression profiles (GEPs) are then generated from the biological samples based on total RNA according to well-established methods. Briefly, a typical method involves isolating total RNA from the biological sample, amplifying the RNA, synthesizing cDNA, labeling the cDNA with a detectable label, hybridizing the cDNA with a genomic array, such as the Affymetrix U133 GeneChip, and determining binding of the labeled cDNA with the genomic array by measuring the intensity of the signal from the detectable label bound to the array. See, e.g., the methods described in Lu, et al., Chen, et al. and Golub, et al., supra, and the references cited therein, which are incorporated herein by reference. The resulting expression data are input into a database.

mRNAs in the tissue samples can be analyzed using commercially available or customized probes or oligonucleotide arrays, such as cDNA or oligonucleotide arrays. The use of these arrays allows for the measurement of steady-state mRNA levels of thousands of genes simultaneously, thereby presenting a powerful tool for identifying effects such as the onset, arrest or modulation of uncontrolled cell proliferation. Hybridization and/or binding of the probes on the arrays to the nucleic acids of interest from the cells can be determined by detecting and/or measuring the location and intensity of the signal received from the labeled probe or used to detect a DNA/RNA sequence from the sample that hybridizes to a nucleic acid sequence at a known location on the microarray. The intensity of the signal is proportional to the quantity of cDNA or mRNA present in the sample tissue. Numerous arrays and techniques are available and useful. Methods for determining gene and/or protein expression in sample tissues are described, for example, in U.S. Pat. No. 6,271,002; U.S. Pat. No. 6,218,122; U.S. Pat. No. 6,218,114; and U.S. Pat. No. 6,004,755; and in Wang et al., J. Clin. Oncol., 22(9):1564-1671 (2004); Golub et al, (supra); and Schena et al., Science, 270:467-470 (1995); all of which are incorporated herein by reference.

The gene analysis aspect may interrogate gene expression as well as insertion/deletion data. As a first step, RNA is isolated from the tissue samples and labeled. Parallel processes are run on the sample to develop two sets of data: (1) over-/under-expression of genes based on mRNA levels; and (2) chromosomal insertion/deletion data. These two sets of data are then correlated by means of an algorithm. Over-/under-expression of the genes in each tissue sample are compared to gene expression in the normal samples and other control samples, and a subset of genes that are differentially expressed in the diseased tissue is identified. Preferably, levels of up- and down-regulation are distinguished based on fold changes of the intensity measurements of hybridized microarray probes. A difference of about 2.0 fold or greater is preferred for making such distinctions, or a p-value of less than about 0.05. That is, before a gene is said to be differentially expressed in diseased or suspected diseased versus normal cells, the diseased cell is found to yield at least about 2 times greater or less intensity of expression than the normal cells. Generally, the greater the fold difference (or the lower the p-value), the more preferred is the gene for use as a diagnostic or prognostic tool. Genes identified for the gene signatures of the present invention have expression levels that result in the generation of a signal that is distinguishable from those of the normal or non-modulated genes by an amount that exceeds background using clinical laboratory instrumentation.

Statistical values can be used to confidently distinguish modulated from non-modulated genes and noise. Statistical tests can identify the genes most significantly differentially expressed between diverse groups of samples. The Student's t-test is an example of a robust statistical test that can be used to find significant differences between two groups. The lower the p-value, the more compelling the evidence that the gene is showing a difference between the different groups. Nevertheless, since microarrays allow measurement of more than one gene at a time, tens of thousands of statistical tests may be run at one time. Because of this, it is unlikely to observe small p-values just by chance, and adjustments using a Sidak correction or similar step as well as a randomization/permutation experiment can be made. A p-value less than about 0.05 by the t-test is evidence that the expression level of the gene is significantly different. More compelling evidence is a p-value less than about 0.05 after the Sidak correction is factored in. For a large number of samples in each group, a p-value less than about 0.05 after the randomization/permutation test is the most compelling evidence of a significant difference.

Another parameter that can be used to select genes that generate a signal that is greater than that of the non-modulated gene or noise is the measurement of absolute signal difference. Preferably, the signal generated by the differentially expressed genes differs by at least about 20% from those of the normal or non-modulated gene (on an absolute basis). It is even more preferred that such genes produce expression patterns that are at least about 30% different than those of normal or non-modulated genes. For smaller subsets of genes evaluated, such as profiles containing less than 30, less than or about 20 or less than or about 10 genes, the expression patterns may be at least about 40% or at least about 50% different than those of normal or non-modulated genes.

Differential expression analyses can be performed using commercially available arrays, for example, Affymetrix U133 GeneChip® arrays (Affymetrix, Inc.). These arrays have probe sets for the whole human genome immobilized on the chip, and can be used to determine up- and down-regulation of genes in test samples. Other substrates having affixed thereon human genomic DNA or probes capable of detecting expression products, such as those available from Affymetrix, Agilent Technologies, Inc. or Illumina, Inc. also may be used. Currently preferred gene microarrays for use in the present invention include Affymetrix U133 GeneChip® arrays and Agilent Technologies genomic cDNA microarrays. Instruments and reagents for performing gene expression analysis are commercially available. See, e.g., Affymetrix GeneChip® System. The expression data obtained from the analysis then is input into the database.

For chromosomal insertion/deletion analyses, data for the genes of each sample as compared to samples of normal tissue is obtained. The insertion/deletion analysis is generated using an array-based comparative genomic hybridization (“CGH”). Array CGH measures copy-number variations at multiple loci simultaneously, providing an important tool for studying disease and developmental disorders and for developing diagnostic and therapeutic targets. Microchips for performing array CGH are commercially available, e.g., from Agilent Technologies. The Agilent chip is a chromosomal array which shows the location of genes on the chromosomes and provides additional data for the gene signature. The insertion/deletion data once acquired from this testing is also input into the database.

The analyses are carried out on the same samples from the same patients to generate parallel data. The same chips and sample preparation are used to reduce variability.

The expression of certain genes known as “reference genes” “control genes” or “housekeeping genes” also is determined, preferably at the same time, as a means of ensuring the veracity of the expression profile. Reference genes are genes that are consistently expressed in many tissue types, including diseased and normal tissues, and thus are useful to normalize gene expression profiles. See, e.g., Silvia et al., BMC Cancer, 6:200 (2006); Lee et al., Genome Research, 12(2):292-297 (2002); Zhang et al., BMC Mol. Biol., 6:4 (2005). Determining the expression of reference genes in parallel with the genes in the unique gene expression profile provides further assurance that the techniques used for determination of the gene expression profile are working properly. The expression data relating to the reference genes also is input into the database. The following genes may be used as reference genes: beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

### Data Correlation for Gene Expression Profiles

The differential expression data and the insertion/deletion data in the database may be correlated with the clinical outcomes information associated with each subject or biologic sample also in the database by means of an algorithm to determine a gene expression profile for determining or predicting progression as well as recurrence of disease and/or disease-related presentations. Various algorithms are available which are useful for correlating the data and identifying the predictive gene signatures. For example, algorithms such as those identified in Xu et al., A Smooth Response Surface Algorithm For Constructing A Gene Regulatory Network, Physiol. Genomics 11:11-20 (2002), the entirety of which is incorporated herein by reference, may be used for the practice of the embodiments disclosed herein.

Another method for identifying gene expression profiles is through the use of optimization algorithms such as the mean variance algorithm widely used in establishing stock portfolios. One such method is described in detail in the patent application US Patent Application Publication No. 2003/0194734. Essentially, the method calls for the establishment of a set of inputs expression as measured by intensity) that will optimize the return (signal that is generated) one receives for using it while minimizing the variability of the return. The algorithm described in Irizarry et al., Nucleic Acids Res., 31:e15 (2003) also may be used. One useful algorithm is the JMP Genomics algorithm available from JMP Software.

The process of selecting gene expression profiles also may include the application of heuristic rules. Such rules are formulated based on biology and an understanding of the technology used to produce clinical results, and are then applied to output from the optimization method. For example, the mean variance method of gene signature identification can be applied to microarray data for a number of genes differentially expressed in subjects with metabolic syndrome and/or heart failure. Output from the method would be an optimized set of genes that could include some genes that are expressed in peripheral blood as well as in diseased tissue. If samples used in the testing method are obtained from peripheral blood and certain genes differentially expressed in instances of metabolic syndrome and/or heart failure could also be differentially expressed in peripheral blood, then a heuristic rule can be applied in which a portfolio is selected from the efficient frontier excluding those that are differentially expressed in peripheral blood. Other cells, tissues or fluids may also be used for the evaluation of differentially expressed genes, proteins or peptides. Of course, the rule can be applied prior to the formation of the efficient frontier by, for example, applying the rule during data pre-selection.

Other heuristic rules can be applied that are not necessarily related to the biology in question. For example, one can apply a rule that only a certain percentage of the portfolio can be represented by a particular gene or group of genes. Commercially available software such as the Wagner software readily accommodates these types of heuristics (Wagner Associates Mean-Variance Optimization Application). This can be useful, for example, when factors other than accuracy and precision have an impact on the desirability of including one or more genes.

As an example, the algorithm may be used for comparing gene expression profiles for various genes (or portfolios) to ascribe prognoses. The expression profiles (whether at the RNA or protein level) of each of the genes comprising the portfolio are fixed in a medium such as a computer readable medium. This can take a number of forms. For example, a table can be established into which the range of signals (e.g., intensity measurements) indicative of disease is input. Actual patient data can then be compared to the values in the table to determine whether the patient samples are normal or diseased. In a more sophisticated embodiment, patterns of the expression signals (e.g., fluorescent intensity) are recorded digitally or graphically. The gene expression patterns from the gene portfolios used in conjunction with patient samples are then compared to the expression patterns. Pattern comparison software can then be used to determine whether the patient samples have a pattern indicative of recurrence of the disease. Of course, these comparisons can also be used to determine whether the patient is not likely to experience disease recurrence. The expression profiles of the samples are then compared to the profile of a control cell. If the sample expression patterns are consistent with the expression pattern for recurrence of metabolic syndrome and/or heart failure then (in the absence of countervailing medical considerations) the patient is treated as one would treat a relapse patient. If the sample expression patterns are consistent with the expression pattern from the normal/control cell then the patient is diagnosed negative for the syndrome.

A method for analyzing the gene signatures of a patient to determine prognosis of a condition (including, but not limited to metabolic syndrome and/or heart failure) is through the use of a Cox hazard analysis program. The analysis may be conducted using S-Plus software (commercially available from Insightful Corporation). Using such methods, a gene expression profile is compared to that of a profile that confidently represents no instance of the condition being analyzed, or to that of a profile that confidently shows an instance of the condition being analyzed or to that of a profile that confidently shown progression of the condition being analyzed. The Cox hazard model with the established threshold is used to compare the similarity of the two profiles (known relapse versus patient) and then determines whether the patient profile exceeds the threshold. If it does, then the patient is classified as one who will progress to the condition being analyzed and is accorded treatment for the condition or any underlying conditions associated with it. If the patient profile does not exceed the threshold then they are classified as a not likely to progress to the condition being analyzed. Other analytical tools can also be used to answer the same question such as, linear discriminate analysis, logistic regression and neural network approaches. See, e.g., software available from JMP statistical software.

Numerous other well-known methods of pattern recognition are available. The following references provide some examples:

Weighted Voting: Golub, T R., Slonim, D K., Tamaya, P., Huard, C., Gaasenbeek, M., Mesirov, J P., Coller, H., Loh, L., Downing, J R., Caligiuri, M A., Bloomfield, C D., Lander, E S. Molecular classification of cancer: class discovery and class prediction by gene expression monitoring. Science 286:531-537, 1999.

Support Vector Machines: Su, A I., Welsh, J B., Sapinoso, L M., Kern, S G., Dimitrov, P., Lapp, H., Schultz, P G., Powell, S M., Moskaluk, C A., Frierson, H F. Jr., Hampton, G M. Molecular classification of human carcinomas by use of gene expression signatures. Cancer Research 61:7388-93, 2001. Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

K-nearest Neighbors: Ramaswamy, S., Tamayo, P., Rifkin, R., Mukherjee, S., Yeang, C H., Angelo, M., Ladd, C., Reich, M., Latulippe, E., Mesirov, J P., Poggio, T., Gerald, W., Loda, M., Lander, E S., Gould, T R. Multiclass cancer diagnosis using tumor gene expression signatures Proceedings of the National Academy of Sciences of the USA 98:15149-15154, 2001.

Correlation Coefficients: van't Veer L J, Dai H, van de Vijver M J, He Y D, Hart A, Mao M, Peters H L, van der Kooy K, Marton M J, Witteveen A T, Schreiber G J, Kerkhoven R M, Roberts C, Linsley P S, Bernards R, Friend S H. Nature. 2002 Jan. 31; 415(6871):530-6.

The gene expression analysis identifies a gene expression profile (GEP) unique to the samples, that is, those genes which are differentially expressed by the cells. This GEP then is validated, for example, using real-time quantitative polymerase chain reaction (RT-qPCR), which may be carried out using commercially available instruments and reagents, such as those available from Applied Biosystems.

### Determination of Protein Expression Profiles

Not all genes expressed by a cell are translated into proteins, therefore, once a GEP has been identified, it may also be desirable to ascertain whether proteins corresponding to some or all of the differentially expressed genes in the GEP also are differentially expressed by the same cells or tissue. Therefore, protein expression profiles (PEPs) are generated from the same suspect tissue control tissues used to identify the GEPs. PEPs also are used to validate the GEP in other individuals, e.g., diabetic, heart failure or metabolic syndrome patients.

The preferred method for generating PEPs according to the present invention is by immunohistochemistry (IHC) analysis. In this method antibodies specific for the proteins in the PEP are used to interrogate tissue samples from individuals of interest. Other methods for identifying PEPs are known, e.g. in situ hybridization (ISH) using protein-specific nucleic acid probes. See, e.g., Hofer et al., Clin. Can. Res., 11(16):5722 (2005); Volm et al., Clin. Exp. Metas., 19(5):385 (2002). Any of these alternative methods also could be used.

Where tissue samples are available, PEP and GEP may be determined using TMA. Otherwise, serum samples may be used to generate PEP and/or GEP. ELISA assays may be employed using serum samples. For determining the PEPs samples of suspect tissue or serum, affected and normal are obtained from patients. These are the same samples used for identifying the GEP. The tissue samples as well as the positive and negative control samples are arrayed on tissue microarrays (TMAs) to enable simultaneous analysis. TMAs consist of substrates, such as glass slides, on which up to about 1000 separate tissue samples are assembled in array fashion to allow simultaneous histological analysis. The tissue samples may comprise tissue obtained from preserved biopsy samples, e.g., paraffin-embedded or frozen tissues. Techniques for making tissue microarrays are well-known in the art. See, e.g., Simon et al., BioTechniques, 36(1):98-105 (2004); Kallioniemi et al, WO 99/44062; Kononen et al., Nat. Med., 4:844-847 (1998). In one method, a hollow needle is used to remove tissue cores as small as 0.6 mm in diameter from regions of interest in paraffin embedded tissues. The “regions of interest” are those that have been identified by a pathologist as containing the desired diseased or normal tissue. These tissue cores are then inserted in a recipient paraffin block in a precisely spaced array pattern. Sections from this block are cut using a microtome, mounted on a microscope slide and then analyzed by standard histological analysis. Each microarray block can be cut into approximately 100 to approximately 500 sections, which can be subjected to independent tests.

Proteins in the tissue samples may be analyzed by interrogating the TMAs using protein-specific agents, such as antibodies or nucleic acid probes, such as oligonucleotides or aptamers. The tissue arrays used may include tissue selected from, but not limited to, the pancreas, colon, gall bladder, kidney, bladder, adipose tissue and muscle pectoralis. Antibodies are preferred for this purpose due to their specificity and availability. The antibodies may be monoclonal or polyclonal antibodies, antibody fragments, and/or various types of synthetic antibodies, including chimeric antibodies, or fragments thereof. Antibodies are commercially available from a number of sources (e.g., Abcam, Cell Signaling Technology or Santa Cruz Biotechnology), or may be generated using techniques well-known to those skilled in the art. The antibodies typically are equipped with detectable labels, such as enzymes, chromogens or quantum dots, which permit the antibodies to be detected. The antibodies may be conjugated or tagged directly with a detectable label, or indirectly with one member of a binding pair, of which the other member contains a detectable label. Detection systems for use with are described, for example, in the website of Ventana Medical Systems, Inc. Quantum dots are particularly useful as detectable labels. The use of quantum dots is described, for example, in the following references: Jaiswal et al., Nat. Biotechnol., 21:47-51 (2003); Chan et al., Curr. Opin. Biotechnol., 13:40-46 (2002); Chan et al., Science, 281:435-446 (1998).

The use of antibodies to identify proteins of interest in the cells of a tissue, referred to as immunohistochemistry (IHC), is well established. See, e.g., Simon et al., BioTechniques, 36(1):98 (2004); Haedicke et al., BioTechniques, 35(1):164 (2003), which are hereby incorporated by reference. The IHC assay can be automated using commercially available instruments, such as the Benchmark instruments available from Ventana Medical Systems, Inc.

In one embodiment, the TMAs are contacted with antibodies specific for the proteins encoded by the genes identified in the gene expression study as being differentially expressed in subjects whose conditions had progressed to metabolic syndrome and/or heart failure in order to determine expression of these proteins in each type of tissue. The antibodies used to interrogate the TMAs are selected based on the genes having the highest level of differential expression.

### GPEP Assays

The present invention further comprises methods and assays for determining or predicting whether a patient's condition is likely to progress to metabolic syndrome and/or heart failure or whether a patient having an underlying condition associated with metabolic syndrome and/or heart failure is likely to progress to metabolic syndrome. According to one aspect, a formatted IHC assay can be used for determining if a tissue sample exhibits any of a GEP, PEP or GPEPs. The assays may be formulated into kits that include all or some of the materials needed to conduct the analysis, including reagents (antibodies, detectable labels, etc.) and instructions.

Any of the compositions described herein may be comprised in a kit. In a non-limiting example, reagents for the detection of PEPs, GEPs, or GPEPs are included in a kit. In one embodiment, antibodies to one or more of the expression products of the genes of the GPEPs disclosed herein are included. Antibodies may be included to provide concentrations of from about 0.1 μg/mL to about 500 μg/mL, from about 0.1 μg/mL to about 50 μg/mL or from about 1 μg/mL to about 5 μg/mL or any value within the stated ranges. The kit may further include reagents or instructions for creating or synthesizing further probes, labels or capture agents. It may also include one or more buffers, such as a nuclease buffer, transcription buffer, or a hybridization buffer, compounds for preparing a DNA template, cDNA, primers, probes or label, and components for isolating any of the foregoing. Other kits of the invention may include components for making a nucleic acid or peptide array including all reagents, buffers and the like and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial or similar container. The kits of the present invention also will typically include a means for containing the detection reagents, e.g., nucleic acids or proteins or antibodies, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

Kits may also include components that preserve or maintain the compositions that protect against their degradation. Such kits generally will comprise, in suitable means, distinct containers for each individual reagent or solution.

Certain assay methods of the invention comprises contacting a tissue sample from an individual with a group of antibodies specific for some or all of the genes or proteins of a GPEP, and determining the occurrence of up- or down-regulation of these genes or proteins in the sample. The use of TMAs allows numerous samples, including control samples, to be assayed simultaneously.

The method preferably also includes detecting and/or quantitating control or “reference proteins”. Detecting and/or quantitating the reference proteins in the samples normalizes the results and thus provides further assurance that the assay is working properly. In a currently preferred embodiment, antibodies specific for one or more of the following reference proteins are included: beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

In one embodiment, the assay and method comprises determining expression only of the overexpressed genes or proteins in a GPEP. The method comprises obtaining a tissue sample from the patient, determining the gene and/or protein expression profile of the sample, and determining from the gene or protein expression profile.

In one embodiment, the assay and method comprises determining expression only of the overexpressed genes or proteins in the GPEP. The method preferably includes at least one reference protein, which may be selected are beta-actin (ACTB), glyceraldehyde-3-phosphate dehydrogenase (GAPDH), beta glucoronidase (GUSB) as positive controls while negative controls include large ribosomal protein (RPLPO) and/or transferrin receptor (TRFC). Beta actin may be used as the positive control for IHC.

The present invention further comprises a kit containing reagents for conducting an IHC analysis of tissue samples or cells from individuals, e.g., patients, including antibodies specific for at least about two of the proteins in a GPEP and for any reference proteins. The antibodies are preferably tagged with means for detecting the binding of the antibodies to the proteins of interest, e.g., detectable labels. Preferred detectable labels include fluorescent compounds or quantum dots; however other types of detectable labels may be used. Detectable labels for antibodies are commercially available, e.g. from Ventana Medical Systems, Inc.

For tissue arrarys, immunohistochemical methods for detecting and quantitating protein expression in tissue samples are well known. Any method that permits the determination of expression of several different proteins can be used. Such methods can be efficiently carried out using automated instruments designed for immunohistochemical (IHC) analysis. Instruments for rapidly performing such assays are commercially available, e.g., from Ventana Molecular Discovery Systems or Lab Vision Corporation. Methods according to the present invention using such instruments are carried out according to the manufacturer's instructions.

Protein-specific antibodies for use in such methods or assays are readily available or can be prepared using well-established techniques. Antibodies specific for the proteins in the GPEP disclosed herein can be obtained, for example, from Cell Signaling Technology, Inc, Santa Cruz Biotechnology, Inc. or Abcam.

### Immunoassays

The present invention provides for new assays useful in the diagnosis, prognosis and prediction of metabolic syndrome and/or heart failure and the elucidation of clinical management parameters associated with metabolic syndrome and/or heart failure. The immunoassays of the present invention utilize the anti-FASN polyclonal or monoclonal antibodies described herein to specifically bind to FASN in a biological sample. Antibodies to other biomarkers (including, but not limited to USP2A, GSTΩ1, SOD2, KCNE2 and BNP) described herein are also available commercially and may be utilized in immunoassays of other embodiments of the present invention. Any type of immunoassay format may be used, including, without limitation, enzyme immunoassays (EIA, ELISA), radioimmunoassay (RIA), fluoroimmunoassay (FIA), chemiluminescent immunoassay (CLIA), counting immunoassay (CIA), immunohistochemistry (IHC), agglutination, nephelometry, turbidimetry or Western Blot. These and other types of immunoassays are well-known and are described in the literature, for example, in Immunochemistry, Van Oss and Van Regenmortel (Eds), CRC Press, 1994; The Immunoassay Handbook, D. Wild (Ed.), Elsevier Ltd., 2005; and the references disclosed therein.

The preferred assay format for the present invention is the enzyme-linked immunosorbent assay (ELISA) format. ELISA is a highly sensitive technique for detecting and measuring antigens or antibodies in a solution in which the solution is run over a surface to which immobilized antibodies specific to the substance have been attached, and if the substance is present, it will bind to the antibody layer, and its presence is verified and visualized with an application of antibodies that have been tagged or labeled so as to permit detection. ELISAs combine the high specificity of antibodies with the high sensitivity of enzyme assays by using antibodies or antigens coupled to an easily assayed enzyme that possesses a high turnover number such as alkaline phosphatase (AP) or horseradish peroxidase (HRP), and are very useful tools both for determining antibody concentrations (antibody titer) in sera as well as for detecting the presence of antigen.

There are many different types of ELISAs; the most common types include “direct ELISA,” “indirect ELISA,” “sandwich ELISA” and cell-based ELISA (C-ELISA). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized the detection antibody is added, forming a complex with the antigen. The detection antibody can be covalently linked to an enzyme, or can itself be detected by a secondary antibody which is linked to an enzyme through bioconjugation. Between each step the plate typically is washed with a mild detergent solution to remove any proteins or antibodies that are not specifically bound. After the final wash step the plate is developed by adding an enzymatic substrate tagged with a detectable label to produce a visible signal, which indicates the quantity of antigen in the sample.

In a typical microtiter plate sandwich immunoassay, an antibody (“capture antibody”) is adsorbed or immobilized onto a substrate, such as a microtiter plate. Monoclonal antibodies are preferred as capture antibodies due to their greater specificity, but polyclonal antibodies also may be used. When the test sample is added to the plate, the antibody on the plate will bind the target antigen from the sample, and retain it in the plate. When a second antibody (“detection antibody”) or antibody pair is added in the next step, it also binds to the target antigen (already bound to the monoclonal antibody on the plate), thereby forming an antigen ‘sandwich’ between the two different antibodies.

This binding reaction can then be measured by radio-isotopes, as in a radio-immunoassay format (RIA); by enzymes, as in an enzyme immunoassay format (EIA or ELISA); or other detectable label, attached to the detection antibody. The label generates a color signal proportional to the amount of target antigen present in the original sample added to the plate. Depending on the immunoassay format, the degree of color can be detected and measured with the naked eye (as with a home pregnancy test), a scintillation counter (for an RIA), or with a spectrophotometric plate reader (for an EIA or ELISA).

### Assay Steps

Step 1: Capture antibodies are adsorbed onto the well of a plastic microtiter plate (no sample added);

Step 2: A test sample (such as human serum) is added to the well of the plate, under conditions sufficient to permit binding of the target antigen to the capture antibody already bound to the plate, thereby retaining the antigen in the well;

Step 3: Binding of a detection antibody or antibody pair (with enzyme or other detectable moiety attached) to the target antigen (already bound to the capture antibody on the plate), thereby forming an antigen “sandwich” between the two different antibodies. The detectable label on the detection antibodies will generate a color signal proportional to the amount of target antigen present in the original sample added to the plate.

In an alternative embodiment, sometimes referred to as an antigen-down immunoassay, the analyte (rather than an antibody) is coated onto a substrate, such as a microtiter plate, and used to bind antibodies found in a sample. When the sample is added (such as human serum), the antigen on the plate is bound by antibodies (IgE for example) from the sample, which are then retained in the well. A species-specific antibody (anti-human IgE for example) labeled with an enzyme such as horse radish peroxidase (HRP) is added next, which, binds to the antibody bound to the antigen on the plate. The higher the signal, the more antibodies there are in the sample.

In another embodiment, an immunoassay may be structured in a competitive inhibition format. Competitive inhibition assays are often used to measure small analytes because competitive inhibition assays only require the binding of one antibody rather than two as is used in standard ELISA formats. In a sequential competitive inhibition assay, the sample and conjugated analyte are added in steps similar to a sandwich assay, while in a classic competitive inhibition assay, these reagents are incubated together at the same time.

In a typical sequential competitive inhibition assay format, a capture antibody is coated onto a substrate, such as a microtiter plate. When the sample is added, the capture antibody captures free analyte out of the sample. In the next step, a known amount of analyte labeled with a detectable label, such as an enzyme or enzyme substrate, added. The labeled analyte also attempts to bind to the capture antibody adsorbed onto the plate, however, the labeled analyte is inhibited from binding to the capture antibody by the presence of previously bound analyte from the sample. This means that the labeled analyte will not be bound by the monoclonal on the plate if the monoclonal has already bound unlabeled analyte from the sample. The amount of unlabeled analyte in the sample is inversely proportional to the signal generated by the labeled analyte. The lower the signal, the more unlabeled analyte there is in the sample. A standard curve can be constructed using serial dilutions of an unlabeled analyte standard. Subsequent sample values can then be read off the standard curve as is done in the sandwich ELISA formats. The classic competitive inhibition assay format requires the simultaneous addition of labeled (conjugated analyte) and unlabeled analyte (from the sample). Both labeled and unlabeled analyte then compete simultaneously for the binding site on the monoclonal capture antibody on the plate. Like the sequential competitive inhibition format, the colored signal is inversely proportional to the concentration of unlabeled target analyte in the sample. Detection of labeled analyte can be read on a microtiter plate reader.

In addition to microtiter plates, immunoassays are also may be configured as rapid tests, such as a home pregnancy test. Like microtiter plate assays, rapid tests use antibodies to react with antigens and can be developed as sandwich formats, competitive inhibition formats, and antigen-down formats. With a rapid test, the antibody and antigen reagents are bound to porous membranes, which react with positive samples while channeling excess fluids to a non-reactive part of the membrane. Rapid immunoassays commonly come in two configurations: a lateral flow test where the sample is simply placed in a well and the results are read immediately; and a flow through system, which requires placing the sample in a well, washing the well, and then finally adding an analyte-detectable label conjugate and the result is read after a few minutes. One sample is tested per strip or cassette. Rapid tests are faster than microtiter plate assays, require little sample processing, are often cheaper, and generate yes/no answers without using an instrument. However, rapid immunoassays are not as sensitive as plate-based immunoassays, nor can they be used to accurately quantitate an analyte.

In one embodiment, a technique for use in the present invention to detect the amount of a biomarker (including, but not limited to FASN, USP2A, GSTΩ1, SOD2, KCNE2 or BNP) in circulating cells is the sandwich ELISA, in which highly specific monoclonal antibodies are used to detect sample antigen. The sandwich ELISA method comprises the following general steps:

Prepare a surface to which a known quantity of capture antibody is bound;

(Optionally) block any non-specific binding sites on the surface;

Apply the antigen-containing sample to the surface;

Wash the surface, so that unbound antigen is removed;

Apply primary (detection) antibodies that bind specifically to the bound antigen;

Apply enzyme-linked secondary antibodies which are specific to the primary antibodies;

Wash the plate, so that the unbound antibody-enzyme conjugates are removed;

Apply a chemical which is converted by the enzyme into a detectable (e.g., color or fluorescent or electrochemical) signal; and

Measure the absorbance or fluorescence or electrochemical signal to determine the presence and quantity of antigen.

In an alternate embodiment, the primary antibody (step 5) is linked to an enzyme; in this embodiment, the use of a secondary antibody conjugated to an enzyme (step 6) is not necessary if the primary antibody is conjugated to an enzyme. However, use of a secondary-antibody conjugate avoids the expensive process of creating enzyme-linked antibodies for every antigen one might want to detect. By using an enzyme-linked antibody that binds the Fc region of other antibodies, this same enzyme-linked antibody can be used in a variety of situations. The major advantage of a sandwich ELISA is the ability to use crude or impure samples and still selectively bind any antigen that may be present. Without the first layer of “capture” antibody, any proteins in the sample (including serum proteins) may competitively adsorb to the plate surface, lowering the quantity of antigen immobilized.

In one embodiment of the present invention, a solid phase substrate, such as a microtiter plate or strip, is treated in order to fix or immobilize a capture antibody to the surface of the substrate. The material of the solid phase is not particularly limited as long as it is a material of a usual solid phase used in immunoassays. Examples of such material include polymer materials such as latex, rubber, polyethylene, polypropylene, polystyrene, a styrene-butadiene copolymer, polyvinyl chloride, polyvinyl acetate, polyacrylamide, polymethacrylate, a styrene-methacrylate copolymer, polyglycidyl methacrylate, an acrolein-ethyleneglycol dimethacrylate copolymer, polyvinylidene difluoride (PVDF), and silicone; agarose; gelatin; red blood cells; and inorganic materials such as silica gel, glass, inert alumina, and magnetic substances. These materials may be used singly or in combination of two or more thereof.

The form of the solid phase is not particularly limited insofar as the solid phase is in the form of a usual solid phase used in immunoassays, for example in the form of a microtiter plate, a test tube, beads, particles, and nanoparticles. The particles include magnetic particles, hydrophobic particles such as polystyrene latex, copolymer latex particles having hydrophilic groups such as an amino group and a carboxyl group on the surfaces of the particles, red blood cells and gelatin particles. The solid phase is preferably a microtiter plate or strip, such as those available from Cell Signaling Technology, Inc.

In one embodiment, the capture antibody is one or more monoclonal anti-FASN antibodies described herein. Where microtiter plates or strips are used, the capture antibody is immobilized within the wells. Techniques for coating and/or immobilizing proteins to solid phase substrates are known in the art, and can be achieved, for example, by a physical adsorption method, a covalent bonding method, an ionic bonding method, or a combination thereof. See, e.g., W. Luttmann et al., Immunology, Ch. 4.3.1 (pp. 92-94), Elsevier, Inc. (2006) and the references cited therein. For example, when the binding substance is avidin or streptavidin, a solid phase to which biotin was bound can be used to fix avidin or streptavidin to the solid phase. The amounts of the capture antibody, the detection antibody and the solid phase to be used can also be suitably established depending on the antigen to be measured, the antibody to be used, and the type of the solid phase or the like. Protocols for coating microtiter plates with capture antibodies, including tools and methods for calculating the quantity of capture antibody, are described for example, on the websites for Immunochemistry Technologies, LLC (Bloomington, Minn.) and Meso Scale Diagnostics, LLC (Gaithersburg, Md.).

The detection antibody can be any anti-FASN antibody. Anti-FASN antibodies are commercially available, for example, from Cell Signaling Technologies, Inc., Santa Cruz Biotechnology, EMD Biosciences, and others. In one embodiment, the detection antibody may be directly conjugated with a detectable label, or an enzyme. If the detection antibody is not conjugated with a detectable label or an enzyme, then a labeled secondary antibody that specifically binds to the detection antibody is included. Such detection antibody “pairs” are commercially available, for example, from Cell Signaling Technologies, Inc.

Techniques for labeling antibodies with detectable labels are well-established in the art. As used herein, the term “detectable label” refers to a composition detectable by spectroscopic, photochemical, biochemical, immunochemical, or chemical means. The detectable label can be selected, e.g., from a group consisting of radioisotopes, fluorescent compounds, chemiluminescent compounds, enzymes, and enzyme co-factors, or any other labels known in the art. See, e.g., Zola, Monoclonal Antibodies: A Manual of Techniques, pp. 147-158 (CRC Press, Inc. 1987). A detectable label can be attached to the subject antibodies and is selected so as to meet the needs of various uses of the method which are often dictated by the availability of assay equipment and compatible immunoassay procedures. Appropriate labels include, without limitation, radionuclides, enzymes (e.g., alkaline phosphatase, horseradish peroxidase, luciferase, or β-galactosidase), fluorescent moieties or proteins (e.g., fluorescein, rhodamine, phycoerythrin, GFP, or BFP), or luminescent moieties (e.g., Evidot® quantum dots supplied by Evident Technologies, Troy, N.Y., or Qdot™ nanoparticles supplied by the Quantum Dot Corporation, Palo Alto, Calif.).

Preferably, the sandwich immunoassay of the present invention comprises the step of measuring the labeled secondary antibody, which is bound to the detection antibody, after formation of the capture antibody-antigen-detection antibody complex on the solid phase. The method of measuring the labeling substance can be appropriately selected depending on the type of the labeling substance. For example, when the labeling substance is a radioisotope, a method of measuring radioactivity by using a conventionally known apparatus such as a scintillation counter can be used. When the labeling substance is a fluorescent substance, a method of measuring fluorescence by using a conventionally known apparatus such as a luminometer can be used.

When the labeling substance is an enzyme, a method of measuring luminescence or coloration by reacting an enzyme substrate with the enzyme can be used. The substrate that can be used for the enzyme includes a conventionally known luminescent substrate, calorimetric substrate, or the like. When an alkaline phosphatase is used as the enzyme, its substrate includes chemilumigenic substrates such as CDP-start (4-chloro-3-(methoxyspiro(1,2-dioxetane-3,2′-(5′-chloro)tricyclo[3.3.1.1.-sup.3.7]decane)-4-yl)disodium phenylphosphate) and CSPD® (3-(4-methoxyspiro(1,2-dioxetane-3,2-(5′-chloro)tricyclo[3.3.1.1.sup.3.7]-decane)-4-yl)disodium phenylphosphate) and colorimetric substrates such as p-nitrophenyl phosphate, 5-bromo-4-chloro-3-indolyl-phosphoric acid (BCIP), 4-nitro blue tetrazolium chloride (NBT), and iodonitro tetrazolium (INT). These luminescent or calorimetric substrates can be detected by a conventionally known spectrophotometer, luminometer, or the like.

In one embodiment, the detectable labels comprise quantum dots (e.g., Evidot® quantum dots supplied by Evident Technologies, Troy, N.Y., or Qdot™ nanoparticles supplied by the Quantum Dot Corporation, Palo Alto, Calif.). Techniques for labeling proteins, including antibodies, with quantum dots are known. See, e.g., Goldman et al., Phys. Stat. Sol., 229(1): 407-414 (2002); Zdobnova et al., J. Biomed. Opt., 14(2):021004 (2009); Lao et al., JACS, 128(46):14756-14757 (2006); Mattoussi et al., JACS, 122(49):12142-12150 (2000); and Mason et al., Methods in Molecular Biology: NanoBiotechnology Protocols, 303:35-50 (Springer Protocols, 2005). Quantum-dot antibody labeling kits are commercially available, e.g., from Invitrogen (Carlsbad, Calif.) and Millipore (Billerica, Mass.).

The sandwich immunoassay of the present invention may comprise one or more washing steps. By washing, the unreacted reagents can be removed. For example, when the solid phase comprises a strip of microtiter wells, a washing substance or buffer is contacted with the wells after each step. Examples of the washing substance that can be used include 2-[N-morpholino]ethanesulfonate buffer (MES), or phosphate buffered saline (PBS), etc. The pH of the buffer is preferably from about pH 6.0 to about pH 10.0. The buffer may contain a detergent or surfactant, such as Tween 20.

The sandwich immunoassay can be carried out under typical conditions for immunoassays. The typical conditions for immunoassays comprise those conditions under which the pH is about 6.0 to 10.0 and the temperature is about 30 to 45° C. The pH can be regulated with a buffer, such as phosphate buffered saline (PBS), a triethanolamine hydrochloride buffer (TEA), a Tris-HCl buffer or the like. The buffer may contain components used in usual immunoassays, such as a surfactant, a preservative and serum proteins. The time of contacting the respective components in each of the respective steps can be suitably established depending on the antigen to be measured, the antibody to be used, and the type of the solid phase or the like.

### SNP Detection

For detection of SNPs, DNA may be extracted from whole blood and genotyping may be performed with iPLEX (Sequenon, San Diego, Calif.) matrix-assisted laser desorption/ioninzation-time of flight mass spectrometry technology. Previously identified SNPs may be obtained using the HapMap database from NCBI and the web-based tagger application from the Broad Institute at Harvard University. Allele frequency may be selected along a continuum depending on the degree of minor allele frequency desired in the analysis.

### Kits

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from patient samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, and the like.

The methods provided by the present invention may also be automated in whole or in part. In one embodiment, the invention further provides kits for performing an immunoassay using the FASN antibodies of the present invention.

All aspects of the present invention may also be practiced such that a limited number of additional genes that are co-expressed with the disclosed genes (e.g., one or more genes from the GPEPs or FASN), for example as evidenced by high Pearson correlation coefficients, are included in a prognostic or predictive tests in addition to and/or in place of disclosed genes.

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below.

### DEFINITIONS

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and most often coding sequences necessary for producing a polypeptide or precursor. Genes, however, may not be translated and instead code for regulatory or structural RNA molecules. Genes include any variants or isoforms, especially those comprising even a single nucleotide polymorphism (SNP).

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The terms “gene expression profile” or “GEP” or “gene signature” refer to a single gene or group of genes expressed by a particular cell or tissue type wherein presence of the gene(s) or transcriptional products thereof, taken individually (as with a single gene marker) or together or the differential expression of such, is indicative/predictive of a certain condition. GEPs include both single gene markers and multi-gene groups.

The phrase “single-gene marker” or “single gene marker” refers to a single gene (including all variants of the gene as well as single nucleotide polymorphisms (SNPs)) expressed by a particular cell or tissue type wherein presence of the gene or transcriptional products thereof, taken individually the differential expression of such, is indicative/predictive of a certain condition.

The phrase “gene-protein expression profile “GPEP” as used herein refers to the group of genes and proteins expressed by a particular cell or tissue type wherein presence of the genes and the proteins, taken together or the differential expression of such, is indicative/predictive of a certain condition. GPEPs are comprised of one or more sets of GEPs and PEPs.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

The term “complementary” as it relates to nucleic acids refers to hybridization or base pairing between nucleotides or nucleic acids, such as, for example, between the two strands of a double-stranded DNA molecule or between an oligonucleotide probe and a target are complementary.

As used herein, an “expression product” is a biomolecule, such as a protein or mRNA, which is produced when a gene in an organism is expressed. An expression product may comprise post-translational modifications. The polypeptide of a gene may be encoded by a full length coding sequence or by any portion of the coding sequence.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of compositions, such as antibodies, which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the polypeptide sequences of the invention (e.g., at the N-terminal or C-terminal ends). Sequence tags can be used for polypeptide purification or localization. Lysines can be used to increase solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants,” when referring to proteins, are those with one or more amino acids in the native or starting amino acid sequence removed. Ordinarily, deletional variants will have one or more amino acids deleted in a particular region of the molecule.

“Covalent derivatives,” when referring to proteins, include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein, the term “peripheral blood mononuclear cell” or “PBMC” refers to mononuclear cells that circulate in the blood. Such cells include, but are not limited to monocytes, T-cells, B-cells and natural killer cells.

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus [terminated by an amino acid with a free carboxyl group (COOH)]. Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

The term “protein expression” refers to the process by which a nucleic acid sequence undergoes translation such that detectable levels of the amino acid sequence or protein are expressed.

The terms “protein expression profile” or “PEP” or “protein expression signature” refer to a group of proteins expressed by a particular cell or tissue type (e.g., neuron, coronary artery endothelium, or diseased tissue), wherein presence of the proteins taken individually (as with a single protein marker) or together or the differential expression of such proteins, is indicative/predictive of a certain condition.

The phrase “single-protein marker” or “single protein marker” refers to a single protein (including all variants of the protein) expressed by a particular cell or tissue type wherein presence of the protein or translational products of the gene encoding said protein, taken individually the differential expression of such, is indicative/predictive of a certain condition.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “array” and “microarray” refer to any type of regular arrangement of objects usually in rows and columns. As it relates to the study of gene and/or protein expression, arrays refer to an arrangement of probes (often oligonucleotide or protein based) or capture agents anchored to a surface which are used to capture or bind to a target of interest. Targets of interest may be genes, products of gene expression, and the like. The type of probe (nucleic acid or protein) represented on the array is dependent on the intended purpose of the array (e.g., to monitor expression of human genes or proteins). The oligonucleotide- or protein-capture agents on a given array may all belong to the same type, category, or group of genes or proteins. Genes or proteins may be considered to be of the same type if they share some common characteristics such as species of origin (e.g., human, mouse, rat); disease state (e.g., cancer, diabetes); structure or functions (e.g., protein kinases, tumor suppressors); or same biological process (e.g., apoptosis, signal transduction, cell cycle regulation, proliferation, differentiation). For example, one array type may be a “cancer array” in which each of the array oligonucleotide- or protein-capture agents correspond to a gene or protein associated with a cancer. An “epithelial array” may be an array of oligonucleotide- or protein-capture agents corresponding to unique epithelial genes or proteins. Similarly, a “cell cycle array” may be an array type in which the oligonucleotide- or protein-capture agents correspond to unique genes or proteins associated with the cell cycle.

The terms “immunohistochemical” or as abbreviated “IHC” as used herein refer to the process of detecting antigens (e.g., proteins) in a biologic sample by exploiting the binding properties of antibodies to antigens in said biologic sample.

The term “immunoassay” refers to a test that uses the binding of antibodies to antigens to identify and measure certain substances. Immunoassays often are used to diagnose disease, and test results can provide information about a disease that may help in planning treatment. An immunoassay takes advantage of the specific binding of an antibody to its antigen. Monoclonal antibodies are often used as they usually bind only to one site of a particular molecule, and therefore provide a more specific and accurate test, which is less easily confused by the presence of other molecules. The antibodies used must have a high affinity for the antigen of interest, because a very high proportion of the antigen must bind to the antibody in order to ensure that the assay has adequate sensitivity.

The term “PCR” or “RT-PCR”, abbreviations for polymerase chain reaction technologies, as used here refer to techniques for the detection or determination of nucleic acid levels, whether synthetic or expressed.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein in diseased tissues or cells versus normal adjacent tissue. For example, a differentially expressed gene may have its expression activated or completely inactivated in normal versus disease conditions, or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions, but is not detectable in both. Stated another way, a gene or protein is differentially expressed when expression of the gene or protein occurs at a higher or lower level in the diseased tissues or cells of a patient relative to the level of its expression in the normal (disease-free) tissues or cells of the patient and/or control tissues or cells.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

The term “complementary” as it relates to arrays refers to the topological compatibility or matching together of the interacting surfaces of a probe molecule and its target. The target and its probe can be described as complementary, and furthermore, the contact surface characteristics are complementary to each other.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE, etc.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. This type of antibodies is produced by the daughter cells of a single antibody-producing hybridoma. A monoclonal antibody typically displays a single binding affinity for any epitope with which it immunoreacts.

The modifier “monoclonal” indicates the character of the antibody as being obtained from a substantially homogeneous population of antibodies, and is not to be construed as requiring production of the antibody by any particular method. Monoclonal antibodies recognize only one type of antigen The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

A monoclonal antibody may contain an antibody molecule having a plurality of antibody combining sites, each immunospecific for a different epitope, e.g., a bispecific monoclonal antibody. Monoclonal antibodies may be obtained by methods known to those skilled in the art. Kohler and Milstein (1975), Nature, 256:495-497; U.S. Pat. No. 4,376,110; Ausubel et al. (1987, 1992), eds., Current Protocols in Molecular Biology, Greene Publishing Assoc. and Wiley Interscience, N.Y.; Harlow and Lane (1988), Antibodies: A Laboratory Manual, Cold Spring Harbor Laboratory; Colligan et al. (1992, 1993), eds., Current Protocols in Immunology, Greene Publishing Assoc. and Wiley Interscience, N.Y.; Iyer et al., Ind. J. Med. Res., (2000), 123:561-564.

An “antibody preparation” is meant to embrace any composition in which an antibody may be present, e.g., a serum (antiserum).

Antibodies may be labeled with detectable labels by one of skill in the art. The label can be a radioisotope, fluorescent compound, chemiluminescent compound, enzyme, or enzyme co-factor, or any other labels known in the art. In some aspects, the antibody that binds to an entity one wishes to measure (the primary antibody) is not labeled, but is instead detected by binding of a labeled secondary antibody that specifically binds to the primary antibody.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. The antibodies of the invention can be from any animal origin including birds and mammals. Preferably, the antibodies are of human, murine (e.g., mouse and rat), donkey, sheep, rabbit, goat, guinea pig, camel, horse, or chicken origin.

Multispecific antibodies can be specific for different epitopes of a peptide of the present invention, or can be specific for both a peptide of the present invention, and a heterologous epitope, such as a heterologous peptide or solid support material. See, e.g., WO 93/17715; WO 92/08802; WO 91/00360; WO 92/05793; Tutt et al., 1991, J. Immunol., 147:60-69; U.S. Pat. Nos. 4,474,893; 4,714,681; 4,925,648; 5,573,920; 5,601,819; and Kostelny et al., 1992, J. Immunol., 148:1547-1553. For example, the antibodies may be produced against a peptide containing repeated units of a FASN peptide sequence of the invention, or they may be produced against a peptide containing two or more FASN peptide sequences of the invention, or the combination thereof.

Moreover, antibodies can also be prepared from any region of the FASN peptides of the invention. In addition, if a polypeptide is a receptor protein, antibodies can be developed against an entire receptor or portions of the receptor, for example, an intracellular domain, an extracellular domain, the entire transmembrane domain, specific transmembrane segments, any of the intracellular or extracellular loops, or any portions of these regions. Antibodies can also be developed against specific functional sites, such as the site of ligand binding, or sites that are glycosylated, phosphorylated, myristylated, or amidated, for example.

By “amplification” is meant production of multiple copies of a target nucleic acid that contains at least a portion of an intended specific target nucleic acid sequence (FASN, USP2a, GSTΩ1, SOD2, KCNE2, BNP etc). The multiple copies may be referred to as amplicons or amplification products. Preferably, the amplified target contains less than the complete target gene sequence (introns and exons) or an expressed target gene sequence (spliced transcript of exons and flanking untranslated sequences). For example, FASN-specific amplicons may be produced by amplifying a portion of the FASN target polynucleotide by using amplification primers which hybridize to, and initiate polymerization from, internal positions of the FASN target polynucleotide. Preferably, the amplified portion contains a detectable target sequence which may be detected using any of a variety of well known methods.

By “primer” or “amplification primer” is meant an oligonucleotide capable of binding to a region of a target nucleic acid or its complement and promoting nucleic acid amplification of the target nucleic acid. In most cases a primer will have a free 3′ end which can be extended by a nucleic acid polymerase. All amplification primers include a base sequence capable of hybridizing via complementary base interactions either directly with at least one strand of the target nucleic acid or with a strand that is complementary to the target sequence. Amplification primers serve as substrates for enzymatic activity that produces a longer nucleic acid product.

A “target-binding sequence” of an amplification primer is the portion that determines target specificity because that portion is capable of annealing to a target nucleic acid strand or its complementary strand. The complementary target sequence to which the target-binding sequence hybridizes is referred to as a primer-binding sequence.

By “detecting” an amplification product is meant any of a variety of methods for determining the presence of an amplified nucleic acid, such as, for example, hybridizing a labeled probe to a portion of the amplified product. In one embodiment, a labeled probe is an oligonucleotide that specifically binds to another sequence and contains a detectable group which may be, for example, a fluorescent moiety, a chemiluminescent moiety, a radioisotope, biotin, avidin, enzyme, enzyme substrate, or other reactive group.

By “nucleic acid amplification conditions” is meant environmental conditions including salt concentration, temperature, the presence or absence of temperature cycling, the presence of a nucleic acid polymerase, nucleoside triphosphates, and cofactors which are sufficient to permit the production of multiple copies of a target nucleic acid or its complementary strand using a nucleic acid amplification method. Many well-known methods of nucleic acid amplification require thermocycling to alternately denature double-stranded nucleic acids and hybridize primers.

The term “biomarker” as used herein refers to a substance indicative of a biological state. According to the present invention, biomarkers include the GPEPs, PEPs, GEPs, as well as the single components or combinations thereof. Biomarkers according to the present invention also include any compounds or compositions which are used to identify or signal the presence of one or more members of the GPEPs, PEPs, GEPs, or combinations thereof disclosed herein. For example, an antibody created to bind to any of the proteins identified as a member of a PEP herein, may be considered useful as a biomarker, although the antibody itself is a secondary indicator.

The term “biological sample” or “biologic sample” refers to a sample obtained from an organism (e.g., a human patient) or from components (e.g., cells) or from body fluids (e.g., blood, serum, sputum, urine, etc) of an organism. The sample may be of any biological tissue, organ, organ system or fluid. The sample may be a “clinical sample” which is a sample derived from a patient. Such samples include, but are not limited to, sputum, blood, serum, blood cells (e.g., white cells), peripheral blood mononuclear cells (PBMCs), amniotic fluid, plasma, semen, bone marrow, and tissue or core, fine or punch needle biopsy samples, urine, peritoneal fluid, and pleural fluid, or cells therefrom. Biological samples may also include sections of tissues such as frozen sections taken for histological purposes. A biological sample may also be referred to as a “patient sample.”

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “syndrome” refers to a collection of symptoms or conditions characterizing a particular clinical etiology. For example, metabolic syndrome is characterized by a collection of physiologic health parameters.

As used herein, the term “body mass index” refers to a number calculated from a subject's weight and height that correlates with the level of body fat of a given subject. This value is obtained from a subject by dividing the weight of the subject in kilograms by (height) in meters. BMI values are interpreted as follows: below 18.5—underweight; 18.5-24.9—normal; 25.0-29.9 overweight; 30.0 and above—obese.

The term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

The term “clinical management parameter” refers to a metric or variable considered important in the detecting, screening, diagnosing, staging or stratifying patients, or determining the progression of, regression of and/or survival from a disease or condition. Examples of such clinical management parameters include, but are not limited to survival in years, disease related death, early or late recurrence, degree of regression, metastasis, responsiveness to treatment, effectiveness of treatment, the likelihood of progression of a condition, blood pressure, body mass index (BMI), levels of insulin, blood sugar, triglycerides, HDL, LDL, C-reactive protein, as well as biomarker status such as levels of FASN, USP2A, GSTΩ1, SOD2, KCNE2, BNP or other gene or a SNP of FASN, GSTΩ1, SOD2, KCNE2, BNP or USP2A, or any metabolic related gene.

The term “endpoint” means a final stage or occurrence along a path or progression.

As used herein, the term “insulin resistance” refers to a physiological condition wherein cells do not respond or do not properly respond to the biological hormone, insulin. Such responses include, but are not limited to insulin binding, insulin-dependent cell signaling, insulin-dependent gene expression, modulation of sugar uptake, modulation of carbohydrate storage and modulation of lipid metabolism.

As used herein, the term “later stage of HF” refers to a stage of HF that occurs as the condition progresses from a less severe stage to a more severe stage, such as from Stage A to Stage B, Stage C or Stage D, from Stage B to Stage C or Stage D, or from Stage C to Stage D. In some embodiments, the term may be used to refer to either of the two final stages of the condition, Stages C and/or D, in the absence of a reference to a prior stage. As used herein, the term “early stage heart failure” refers to a stage of heart failure that is less severe than a later stage. In some embodiments, early stage heart failure is Stage A, Stage B or Stage C.

The term “treating” as used herein, unless otherwise indicated, means reversing, alleviating, inhibiting the progress of, or preventing, either partially or completely, the symptoms, conditions, or underlying causes of metabolic syndrome. The term “treatment” as used herein, unless otherwise indicated, refers to the act of treating.

The phrase “a method of treating” or its equivalent, when applied to, for example, metabolic syndrome refers to a procedure or course of action that is designed to reduce, eliminate or prevent development or progression individual, or to alleviate the symptoms of a metabolic syndrome. “A method of treating” a disorder does not necessarily mean that the disorder will, in fact, be completely eliminated, that the number of cells or disorder will, in fact, be reduced, or that the symptoms of a disease or other disorder will, in fact, be alleviated. Often, a method of treating will be performed even with a low likelihood of success, but which, given the medical history and estimated survival expectancy of an individual, is nevertheless deemed an overall beneficial course of action.

The term “predicting” means a statement or claim that a particular event will, or is very likely to, occur in the future.

The term “prognosing” means a statement or claim that a particular biologic event will, or is very likely to, occur in the future.

The term “progression” or “disease progression” means the advancement or worsening of or toward a disease or condition.

The term “regression” or “degree of regression” refers to the reversal, either phenotypically or genotypically, of a disease progression. Slowing or stopping of any disease progression may be considered regression.

The term “stratifying” as it relates to patients means the parsing of patients into groups of predicted outcomes along a continuum of from a positive outcome (such as disease free) to moderate or good outcomes (such as improved quality of life or increased survival) to poor outcomes (such as terminal prognosis or death).

As used herein, the term “subject” or “patient” refers to any organism to which an embodiment of the invention may be applied, e.g., for experimental, diagnostic, prophylactic, and/or therapeutic purposes. Typical subjects include animals (e.g., mammals such as mice, rats, rabbits, non-human primates, and humans) and/or plants.

The term “therapeutically effective agent” means a composition that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

The invention is further illustrated by the following non-limiting examples.

### EXAMPLES

### Example 1

### Gene Expression Profile (GEP) Analysis

Gene expression profiles were generated for 216 patients with Type2 Diabetes in clinical study (NucDia1), and 218 patients with Type2 diabetes in clinical study (NucDia2). Expression data from the two studies were normalized together by Robust Microarray Analysis (RMA). This study looked at insulin resistant type 2 diabetics with Metformin response (NucDia1) and insulin sensitive type 2 diabetics with Metformin response (NucDia2). Metrics associated with the two clinical study subsets are shown in Table 1.

TABLE 1
Comparison of two clinical study subsets

| | Study Identifier | Study Identifier |
|---|---|---|
| | (NucDia 1) | (NucDia 2) |
| Type 2 Diabetics | 216 | 218 |
| Gene/Protein/Serum | Yes | Yes |
| biomarker | | |
| based determination | | |
| Patient Setting | Inpatient | Inpatient |
| Number of Patients | 216 | 218 |
| Collection Type | Sera and cDNA | Sera and cDNA |
| | from buffy coat | from buffy coat |
| Insulin Resistant | Resistant | Sensitive |
| or Sensitive | | |
| Gene array type | Affymetrix | Affymetrix |
| | HU133A2 - B | HU133A - B |

Gene expression data from the two studies was obtained via immunohistochemical methodology whereby serum biological samples were obtained from patients. Control samples were also obtained. Gene expression profiles (GEPs) then were generated from the samples based on total RNA according to well-established methods (See Affymetrix GeneChip expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix U133 GeneChip genomic array, and binding of the cDNA to the array was quantified by measuring the intensity of the signal from the detectable cDNA label bound to the array.

### Example 2

### Identification of Single Gene Markers

Gene Ontology (GO) analysis was used as described by Lee H K et al., 2005, “Tool for functional analysis of gene expression data sets,” BMC Bioinformatics, 6: 269; (See also: The Gene Ontology Consortium. “Gene ontology: tool for the unification of biology.” Nat. Genet. May 2000; 25(1):25-9 at http://www.geneontology.org) with 10,000 iterations of the Gene Score Re-sampling Algorithm. A gene network was built using the GeneGo program.

### Example 3

### Multi-Probe-Set Predictive Models

To develop a predictive GPEP (gene-protein expression profile), 21,568 probe sets were filtered by removing (a) probe sets with low expression over all samples; and (b) probe sets with low variance over all samples. This yielded 14,536 probe sets for subsequent analyses. Normalized log 2(intensity) values were centered by subtracting the study-specific mean for each probe set, and rescaled by dividing by the pooled within-study standard deviation for each probe set.

A two-stage model-building approach was used to arrive at the best predictive model.

### Single-Gene Markers

Single-probe-set analyses for dimension reduction were performed. This analysis involves an initial search for probe sets that showed a difference between the two studies in the relationship between expression level and response status, by either logistic regression or linear regression. This yielded 653 probe sets.

### Multi-Gene Markers

A fit was examined with multi-probe-set predictive models. Here, the pre-selected probe sets from the single-probe-set analyses from stage 1 were used as the starting point. Then the initial predictive models to each study were fit separately using a threshold gradient descent (TGD) method for regularized classification. Recursive feature elimination (RFE) was applied to attempt to simplify the models without appreciable loss of predictive accuracy.

The model selection criterion was the mean area under the ROC curve (AUC) from 100 replicates of a 4-fold cross-validation. Then from each RFE model series, here, one per study, the model with maximum difference between the selection criteria for the two studies was selected. The TGD method also was used to build predictive models based on expression of two individual probe sets.

### Example 4

### Single-Gene Markers: Insulin Resistance Vs. Response

Genes with the ten largest signal to noise (S2N) scores among those with a range of at least 2.5 for log 2 (expression intensity) and p-value <0.01 for a t-test of the mean expression difference between Insulin Resistance vs response in type 2 diabetes (T2D).

Signal-to-Noise ratios (S2N) were generated by comparing responders in the trials (the whole data set). S2N was calculated based upon the following formula: S2N=|x1−x2|/(s1+s2); where X1 is the mean for trial i and s1 is the standard deviation for trial i, i=1, 2.

TCF7L2 (Transcription factor 7-like 2 (T-cell specific, HMG-box) also known as TCF7L2 or TCF4) encodes a protein involved in cell signaling. How TCF7L2 affects the development of type 2 diabetes is not completely understood. TCF7L2 has been shown to be involved in the development of pancreatic islets, which contain insulin producing beta cells. Studies suggest that the T version of this SNP is associated with impaired baseline insulin secretion.

The FTO gene (fat mass and obesity associated gene) is a nuclear protein of the AlkB related non-heme iron and 2-oxoglutarate-dependent oxygenase superfamily. Studies in mice and humans indicate a role in nervous and cardiovascular systems and a strong association with body mass index, obesity risk, and type 2 diabetes.

The insulin-like growth factor 2 mRNA binding protein 2 (IGF2BP2) gene encodes a member of the IGF-II mRNA-binding protein (IMP) family. The protein contains several four KH domains and two RRM domains. It functions by binding to the 5′ UTR of the insulin-like growth factor 2 (IGF2) mRNA and regulating IGF2 translation. The results of the analyses are shown in Table 2.

The data reveal that the highest signal to noise ratio, hence correlation, for insulin resistance vs. metformin response in type 2 diabetes are SNPs rs4246444, rs6502051 and rs12949488. All of these SNPs are found in the FASN gene. However, not all FASN SNPs were correlated. Two FASN SNPs showed much lower signal to noise scores as did SNPs for the FTO gene and IGF2BP2, each of which might have been expected to show higher correlations in type 2 diabetics.

TABLE 2
Insulin Resistance vs. Response

| SNP | Description | p Value | S2N |
|---|---|---|---|
| rs4246444 | C > A FASN | 0.00034 | 0.914 |
| rs6502051 | G > T FASN | 0.00071 | 0.812 |
| rs12949488 | G > A FASN | 0.00061 | 0.772 |
| rs7903146 | TCF7L2 | 0.00015 | 0.723 |
| rs9930506 | G Allele FTO | 0.00011 | 0.697 |
| rs8066956 | G > A FASN | 0.00010 | 0.629 |
| rs1127678 | G > A FASN | 0.00028 | 0.621 |
| rs4402960 | T Allele IGF2BP2 | 0.00020 | 0.412 |

### Example 5

### Insulin Resistance Biomarkers

Measurements of FASN, USP2A and FASN/USP2A combination as biomarkers were evaluated and a detection rates determined. The detection rate for insulin resistance was determined for all patients, and for only patients with estimated detection probability>an arbitrary threshold of 0.5 based on USP2A or FASN expression level; where R=True number of detections, N=Total number of patients in subset, Detection Rate=R/N. The results of the analyses are shown in Table 3. It is evident from the data that the use of either FASN or USP2A as a biomarker of insulin resistance shows power; and when combined provides an even slightly higher detection rate.

TABLE 3
Biomarkers of Insulin Resistance

| | | Study Identifier | | |
|---|---|---|---|---|
| | | (NucDia 1) | | |
| Model | Subset | R | N | Detection Rate |
| FASN/USP2A | T2D | 198 | 216 | 0.92 |
| USP2A | T2D | 192 | 216 | 0.89 |
| FASN alone | T2D | 196 | 216 | 0.91 |

### Example 6

### Single-Gene Markers: Insulin Sensitivity Vs. Response

Genes with the ten largest signal to noise (S2N) scores among those with a range of at least 2.5 for log 2(expression intensity) and p-value <0.01 for a t-test of the mean expression difference between Insulin Sensitivity vs response in T2D. The results of the analyses are shown in Table 4.

The data reveal that the highest signal to noise ratio, hence correlation, for insulin sensitivity vs. metformin response in type 2 diabetes is SNPs rs4246444 with other FASN SNPs having lower signal to noise. While the top four SNPs are found in the FASN gene, not all FASN SNPs were strongly correlated. One FASN SNPs showed a much lower signal to noise score as did SNPs for the FTO, IGF2BP2, and TCF7L2 genes, each of which might have been expected to show higher correlations in type 2 diabetics.

TABLE 4
Insulin Sensitivity vs. Response

| SNP | Description | p Value | S2N |
|---|---|---|---|
| rs4246444 | C > A FASN | 0.00039 | 0.834 |
| rs12949488 | G > A FASN | 0.00037 | 0.623 |
| rs8066956 | G > A FASN | 0.00040 | 0.591 |
| rs1127678 | G > A FASN | 0.00039 | 0.561 |
| rs9930506 | G Allele FTO | 0.00018 | 0.513 |
| rs4402960 | T Allele IGF2BP2 | 0.00018 | 0.482 |
| rs7903146 | TCF7L2 | 0.00041 | 0.423 |
| rs6502051 | G > T FASN | 0.00048 | 0.286 |

### Example 7

### Insulin Sensitivity

Measurements of FASN, USP2A and FASN/USP2A combination as biomarkers were evaluated and a detection rates determined. The detection rate for insulin sensitivity was determined for all patients, and for only patients with estimated detection probability>an arbitrary threshold of 0.5 based on USP2A or FASN expression level; where R=True number of detections, N=Total number of patients in subset, Detection Rate=R/N. The results of the analyses are shown in Table 5. It is evident from the data that the use of either FASN or USP2A as a biomarker of insulin sensitivity shows power and when combined provides an even higher detection rate.

TABLE 5
Biomarkers of Insulin Sensitivity

| | | Study Identifier | | |
|---|---|---|---|---|
| | | (NucDia 2) | | |
| Model | Subset | R | N | Detection Rate |
| FASN/USP2A | T2D | 200 | 218 | 0.92 |
| USP2A | T2D | 194 | 218 | 0.88 |
| FASN alone | T2D | 197 | 218 | 0.90 |

### Example 8

### Metabolic Syndrome and Cancer: SNPs as Predictors of Recurrence, Aggressiveness and Cancer Type

In an effort to elucidate the connectivity between metabolic syndrome and cancer, the prevalence and correlation of the presence of certain expression products (genes and proteins) was investigated.

In a study of 546 prostate cancer patient samples (formalin fixed from patients having had radical prostatectomy), SNPs from various chromosomal regions as well as certain gene products were assessed for expression and hazard ratios calculated. The data are shown in Table 6. In the table, HR stands for the “adjusted odds ratio for patients having advanced prostate cancer and a Gleason score of between 5-7”. Values which were found to be statistically significant are labeled with an asterisk “*” and for these data the p value was 0.05 and confidence interval (CI) of 95%. Sample preparation and data analyses were performed according to the methods of Nguyen, et al, J. Clin. Oncol. 28: 2010, 3958-3964).

From the data it can be seen that SNPs mapping to certain regions of chromosome 8 are significantly correlated with higher hazard ratios meaning that these SNPs, alone or in combination, represent potential biomarkers for metabolic syndrome accompanying or associated with prostate cancer.

TABLE 6
Biomarkers

| | Gene/ | | |
|---|---|---|---|
| SNP | Chromosome Region | Genotype | HR 95% CI |
| rs1447295 | 8q24 (region 1) | CC | 0.95 |
| rs6983267 | 8q24 (region 3) | GG | 1.26* |
| rs10505483 | 8q24 (region 2) | CC | 0.98 |
| rs1859962 | 17q24.3 region | GT | 0.94 |
| rs4430796 | TCF2 | AA | 1.29* |
| rs10993994 | 10q11.23 | CT | 1.06* |
| rs7127900 | 11p15.5 | AG | 1.12* |
| rs8102476 | 19q13.2 | CT | 0.99 |
| rs12621278 | ITGA6 | AA | 1.05* |
| rs170021918 | PDLIM5 | CT | 1.0* |
| rs10486567 | JAZF1 | GG | 1.08* |
| rs1512268 | 8p21.2 | CT | 1.04* |

Genes identified with high hazard ratios include TCF2 (now known as HNF1 homeobox B), ITGA6 (integrin, alpha 6), PDLIM5 (PDZ and LIM domain 5), and JAZF1 (JAZF zinc finger 1).

Chromosomal regions which may serve to provide predictive insights include those listed on chromosome 8, 10, and 11.

### Example 9

### Metabolic Syndrome and Cancer: FASN SNPs as Biomarkers

Expanding on the work of Nguyen, a study to determine prostate specific mortality biomarkers was performed. The studies were performed as per Nguyen and the data in Table 7 and partially in Table 8 are those from the Nguyen publication for comparison. (Nguyen, et al, J. Clin. Oncol. 28: 2010, 3958-3964). Shown in Table 8 are the mortality data of the Nguyen study as compared to the outcomes of the present study showing the Hazard Ratios for each SNP identified.

TABLE 7
Nguyen data

| FASN | | |
|---|---|---|
| SNP | Genotype | HR 95% CI |
| rs1127678 | GA | 1.05* |
| rs6502051 | GT | 0.93 |
| rs4246444 | CA | 0.92 |
| rs12949488 | GA | 1.02* |
| rs8066956 | GA | 0.84 |

TABLE 8
Prostate cancer specific mortality

| | | | | HR data |
|---|---|---|---|---|
| FASN | | HR data (95% CI) from Nguyen | | Present |
| SNP | Genotype | Age | Age, BMI, Stage | Study |
| rs6502051 | GG | 1.00 | 1.00 | 1.00 |
| rs6502051 | GT | 0.80 | .071 | 0.88 |
| rs6502051 | TT | 0.66 | .070 | 0.72 |
| | Per Allele | 0.81 | 0.81 | 0.87 |
| rs42446444 | CC | 1.00 | 1.00 | 1.00 |
| rs42446444 | CA | 0.93 | 0.78 | 0.93 |
| rs42446444 | AA | .033 | 0.40 | 0.55 |
| | Per Allele | 0.77 | 0.72 | 0.76 |

### Example 10

### Metabolic Syndrome and Cancer: Non-FASN SNPs as Biomarkers

As in Example 9 and following the procedures of Nguyen, other SNPs were evaluated for their ability to serve as biomarkers of prostate cancer, alone or in association with metabolic syndrome. The data are shown in Tables 9 and 10.

The genes evaluated included FTO (fat mass and obesity associated) gene, MC4R (melanocortin 4 receptor), TMEM18 (transmembrane protein 18), GNPDA2 (glucosamine-6-phosphate deaminase 2; variants of which are associated with obesity), ETV5 (Ets variant 5), BDNF (brain derived neurotrophic factor), SH2B 1 (SH2B adapter protein 1), PCSK1 (proprotein convertase subtilisin/kexin type 1; which regulates insulin biosynthesis), and ATM (ataxia telangiectasia mutated).

TABLE 9
Other Biomarkers

| | Gene/ | | |
|---|---|---|---|
| SNP | Chromosome Region | Genotype | HR 95% CI |
| rs3751812 | FTO | GG | 1.06* |
| rs10871777 | MC4R | AA | 1.09* |
| rs6548238 | TMEM18 | CC | 1.04* |
| rs6548238 | | CT | 1.06* |
| rs6548238 | | TT | 0.94 |
| rs13130484 | GMPDA2 | TT | 0.94 |
| rs13130484 | | CT | 0.96 |
| rs13130484 | | CC | 1.04* |
| rs7647305 | ETV5 | CC | 0.41 |
| rs7647305 | | CT | 0.72 |
| rs7647305 | | TT | 0.94 |
| rs925946 | BDNF | TT | 0.56 |
| rs925946 | | GT | 0.89 |
| rs925946 | | GG | 1.02* |
| rs4788102 | SH2B1 | AA | 0.78 |
| rs4788102 | | AG | 0.83 |
| rs4788102 | | GG | 1.01 |
| rs6232 | PCSK1 | CC | 0.87 |
| rs6232 | | CT | 0.76 |
| rs6232 | | TT | 0.73 |
| rs7566605 | INSIG2 | CC | 0.98 |
| rs7566605 | | CG | 0.96 |
| rs7566605 | | GG | 0.88 |

FTO (fat mass and obesity associated) gene, MC4R (melanocortin 4 receptor), TMEM18 (transmembrane protein 18), GNPDA2 (glucosamine-6-phosphate deaminase 2; variants of which are associated with obesity), ETV5 (Ets variant 5), BDNF (brain derived neurotrophic factor), SH2B1 (SH2B adapter protein 1), PCSK1 (proprotein convertase subtilisin/kexin type 1; which regulates insulin biosynthesis), and ATM (ataxia telangiectasia mutated).

TABLE 10
ATM SNP in metformin response

| | Gene/ | | |
|---|---|---|---|
| SNP | Chromosome Region | Genotype | HR 95% CI |
| rs4585 | ATM | GG | 0.92 |
| | | GT | 1.04* |
| | | TT | 1.00* |

### Example 11

### Preparation of Anti-FASN Monoclonal Antibodies

Anti-FASN antibodies and an immunohistochemical ELISA assay employing the antibodies are disclosed in PCT Publication PCT/US2010/030545 published Oct. 14, 2010, and PCT/US2010/046773 published Mar. 17, 2011, respectively. The contents of each are incorporated here by reference in their entirety.

### Example 12

### ELISA Protocol for Chemiluminescence

Wells are coated with 100 μl/well of coating antibody diluted in appropriate buffer (PBS/PBS-T (0.05% Tween20)). Plates are then incubated overnight at 4° C., covered with plate sealer. The plates are then washed with 300 ul of 5×PBS-T on a Wellwash Versa Plate washer (Thermo). The plates are then blocked with ELISA Blocker Blocking Solution (300 μL1/well) (Thermo) for 2 hr at 23° C. with shaking at 100 rpm in Incubating Microplate Shaker (VWR) covered with a plate sealer. Afterwards, plates are washed with 5×PBS-T (300 μL1/well) on a plate washer. After washing, the plates are tapped on a kimwipe placed on the bench to remove excess liquid.

Standards are prepared in advance and included a 7-point dilution (e.g. in 1% BSA in PBS-T from 500 pg/ml). Once prepared, 100 μl of standards or samples freshly diluted in appropriate buffer (PBS-T, R&D Diluent 7, 18 etc.) are loaded at 23° C. on plate shaker with 100 rpm agitation for 2 hr while covered with plate sealer. Plates are then washed with 5×PBS-T (300 ul/well) on a plate washer.

The detection antibody (100 μl/well; diluted in buffer to appropriate concentration, e.g., in PTS/PBS-T) is incubated for 2 hours at 23° C. on a plate shaker with 100 rpm agitation covered with a plate sealer. The plates were then washed with 5× with PBS-T (300 ul/well) on a plate washer.

The secondary antibody (100 μl/well of appropriate secondary antibody streptavidin-HRP, 1:200 dilution in PBS) is incubated at 23° C. on plate shaker with 100 rpm agitation for 20 min covered with a plate sealer. Alternatively anti-species-HRP antibody at 1:10,000 in PBS for 1 hr at 23° C. on plate shaker with 100 rpm agitation was used. The plates are then washed with 5×PBS-T (300 ul/well) on a plate washer.

The signal is amplified by adding 100 μl/well R&D Gloset Substrate, for 10 min at room temperature in a BioTek FL800x plate reader.

Substrates, which are prepared fresh ahead of time are made by mixing Reagent A (stabilized enhanced luminal) with Reagent B (stabilized hydrogen peroxide) in a 1:2 ratio.

The signal is measured on a BioTek FL800x fluorometer (0.5 s read time) with sensitivity auto-adjusted to the highest point on a standard curve and set to a reading of 100,000.

It should be noted that ELISA Sandwich assays useful in the present invention include those as described in PCT Publication PCT/US2010/046773 published Mar. 17, 2011, the contents of which are incorporated here by reference in its entirety.

### Example 13

### Sample Preparation and In Situ Hybridization Protocols

### A. FFPE Pretreatment Protocol for FISH

The purpose of FFPE pretreatment is to prepare formalin fixed paraffin-embedded (FFPE) tissue sections fixed on positively charged slides for use in fluorescence in situ hybridization (FISH) with CEP and LSI DNA FISH probes. The procedure has been designed to maximize tissue permeability for FISH when using DNA FISH probes.

### Specimen

Formalin fixed paraffin-embedded (FFPE) tissue specimens prepared on microscope slides.

### Reagents and Instrumentation

Preparation involved the use of reagents Provided In Kit (Cat#32-801210). Not provided in the kit are: absolute ethanol (EtoH), Hemo-De Clearing Agent (Scientific Safety Solvents Cat. #HD-150), purified water (distilled or deionized), Coplin jars (16 slides/8 slots capacity maximum), 37° C. and 80° C. water baths (one at 73° C. for the probe assay).

### Paraffin Pretreatment Procedure

Sample Slides Preparation: Samples used are fixed in formalin for between 24-48 hours.

Cut 4-5 μm thick paraffin sections using a microtome.

Float the sections on a purified (i.e., triple distilled) water bath at 40° C.

Mount a section on a positively charged slide.

Air dry the slides.

Bake the slides overnight at 56° C.

Deparaffinizing Slides

Immerse slides in Hemo-De for 5 minutes at ambient temperature.

Repeat step one (1) twice using fresh Hemo-De each time.

Dehydrate slides in 100% EtOH for 1 minute at ambient temperature. Repeat.

Air dry slides for 2-5 minutes, if desired.

### Slide Pretreatment

Immerse slides in Pretreatment Solution at 80° C. for 10 minutes. If necessary, two slides may be placed back-to-back in each slot in the Coplin jar, with one slide placed in each end slot. For the end slides, the side of the slide with the tissue section must face the center of the jar.

Immerse slides in purified water for 3 minutes.

### Protease Pretreatment

Remove slides from the jar of purified water.

Remove excess water by blotting the edges of the slides on a paper towel.

Immerse slides in Protease solution at 37° C. for 15 minutes. (Ensure that the temperature of the buffer is 37±1° C. prior to adding 250 mg (one tube) protease. If necessary, two slides may be placed back-to-back in each slot in the Coplin jar, with one slide placed in each end slot. For the end slides, the side of the slide with the tissue section must face the center of the jar.

Immerse slides in purified water for 3 minutes.

Air dry slides for 2-5 minutes.

### Fixing the Sample (Optional)

Fixation of the sample is performed to minimize tissue loss during sample denaturation. This procedure is highly recommended when processing samples in a denaturation bath format, but is not necessary when processing slides using a Co-denaturation/Hybridization protocol.

Fill one (1) Coplin jar with 50 mL of 10% buffered formalin. Fill three (3) other Coplin jars with 50 mL of 70% ethanol, 85% ethanol and 100% ethanol in each.

Immerse the slides in 10% buffered formalin at ambient temperature for 10 minutes.

Immerse the slides in purified water for 3 minutes.

Air dry slides.

Proceed with the appropriate probe protocol.

### B. Preparation of Metaphase Chromosome Spreads on Microscope Slides for FISH/ISH Analysis

The purpose of this procedure is to prepare human metaphase chromosome spreads and interphase nuclei on microscope slides for cytogenetic analysis and to prepare chromosome preparations for FISH/ISH hybridization procedures.

### Specimen

PHA-stimulated human lymphocytes in 3:1 methanol:glacial acetic acid fixative. The specimens are prepared as described below under “Preparation of Peripheral Blood Cells for Chromosome Analysis”. Table 11 shows reagents and instruments used.

TABLE 11
Reagents and Instrumentation

| Item | Supplier | Catalog No. |
|---|---|---|
| Acetic acid, glacial, 500 mL | VWR | JT9511-5 |
| Methanol, 1.0 L | VWR | JT9049-2 |
| Benchtop Centrifuge, 4x 100 mL capacity | VWR | 53513-800 |
| BD Falcon Centrifuge Tubes, conical, 15 mL | VWR | |
| VWR Superfrost Plus Micro slides | VWR | 48311-703 |
| Glass Pasteur Pipettes with bulb or P-1000 μl | | |
| pipette | | |
| Rectangular Staining Dish With Glass Cover | | |
| Distilled Water | | |
| Kimwipes | | |
| Paper Towels | | |
| Phase Contrast Microscope | | |
| Ethanol Series, 70%, 85%, 100% | | |
| 20 X SSC stock for 2 X SSC | | |
| 37° C. water bath | | |
| PHA-stimulated lymphocyte cell pellet | | |

### Preparation

Fixative:

Methanol:glacial acetic acid, 3:1. Prepare before each use.

Slides:

Label each Superfrost Plus slide accordingly on its frosted surface and place the slides in a rectangular staining dish with glass cover. Fill the dish with distilled water and soak at 4° C. prior to use to chill slides. This can be done days in advance, and slides can be stored at 4° C.

Humidity:

Recommended ambient conditions are 25° C. and 33% humidity.

PHA-Stimulated Lymphocyte Cell Pellet:

Prepare the PHA-stimulated lymphocyte cell pellet in fresh fixative in a 15 mL conical tube. If the pellet was stored after its harvest, centrifuge it at 200×g for 5 minutes. Aspirate the supernatant, and add sufficient fixative to make the cell suspension appear slightly cloudy. Cell concentration varies between cases and should be empirically determined.

Ethanol Series:

Prepare v/v dilutions of 100% ethanol with purified H₂O. Between uses, store tightly covered at ambient temperature. Discard stock solutions after 6 months. Prepare 70%, 85% and 100% ethanol using distilled water in plastic Coplin jars.

2×SSC:

Mix thoroughly 100 mL 20×SSC (pH 5.3) with 850 mL purified H2O. Measure pH and adjust to pH 7.0±0.2 with NaOH. Add purified H2O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

### Procedure

Dropping the cell suspension on slides.

Remove the staining dish containing the Superfrost Plus microscope slides from the 4° C. storage and place on the lab bench.

Using a glass Pasteur pipette with bulb (or P-1000 pipette), gently resuspend the cell pellet in the fixative and set aside in a tube rack.

Remove one microscope slide from the chilled staining jar, holding it by the frosted end. Allow the water to drain from the slide so that a thin film of water remains on the slide surface.

Resuspend the cell pellet using the P-1000 pipette with appropriate tip and then draw 300 ul of the cell suspension.

Holding the slide at an angle (˜45°) expel the cell suspension down the length of the slide, starting at the frosted end. Move the pipette tip across the surface of the slide just below the frosted area from one edge to the other as the suspension is expelled.

Drain the excess cell suspension and fixative from the slide by touching the edges of the slide on a dry paper towel.

Position the slide at an ˜45° C. angle with the cell sample side facing up to dry and allow the fixative to evaporate.

Review the slide preparation with Phase Contrast Microscopy. (See Notes below)

Continue to prepare slides as needed for intended analysis.

Age the slides by placing slides in a coplin jar containing 2×SSC at 37° C. for 30 minutes. Pass slide through an ethanol series, 70%, 85% and 100% for one minute each. Allow to air dry. Alternatively, allow the slides to age at Room Temperature in a slide box for 1 to 4 weeks. (See Note below)

Store slides at −20° C. in dry containers for long-term storage.

Storage of remaining specimen. When an adequate number of slides have been made, store the 15 mL conical tube containing the remaining cell suspension in fixative at −20° C.

### Notes

View the slide preparation with phase contrast microscopy to assess the cell density and metaphase spreading.

If the cell density is too high (more than approximately 100 nuclei per 10× field on the phase contrast microscope), add several drops of fixative to the cell suspension in the 15 mL conical and repeat steps for dropping cells on a new slide.

If the cell density is too sparse (less than ten nuclei per 10× field), centrifuge the 15 mL conical centrifuge tube containing the cells at 200×g for 5 minutes, aspirate the excess fixative, resuspend the pellet in less fixative than added initially, and repeat steps for dropping cells with a new slide.

If there is inadequate spreading so that the majority of chromosomes are indistinguishable, decrease airflow, increase humidity, or decrease temperature to allow the slide to dry slower. If there is over-spreading so that cell boundaries are not distinguishable, increase airflow, decrease humidity, or ambient increase temperature to allow slides to dry faster.

The resulting metaphase cells should have minimal overlaps and no visible cytoplasm, with chromosomes appearing as medium gray to dark gray under phase contrast microscopy.

Aging of cytogenetic preparations denatures the proteins, removes residual water and fixative, and enhances the adherence of the material to the glass. When fresh, non-aged slides are heat denatured they either lose most of their material or their chromosomes become distorted and puffy in appearance. If slides are aged extensively, hybridization efficiency decreases because the chromosomes are too hard.

### C. Preparation of Peripheral Blood Cells for Chromosome Analysis

The purpose of this protocol is to culture and harvest human lymphocytes to determine structural and numerical chromosomal abnormalities and to prepare chromosome preparations for FISH/ISH hybridization procedures. Table 12 shows reagents and instruments used.

### Specimen

Collect 3-5 mL of heparinized whole blood (green top vacutainer tube); sodium heparin is the recommended anticoagulant.

TABLE 12
Reagents and Instrumentation

| Item | Supplier | Catalog No. |
|---|---|---|
| Acetic acid, glacial, 500 mL | VWR | JT9511-5 |
| Methanol, 1.0 L | VWR | JT9049-2 |
| KaryoMAX Colcemid Solution (10 μg/mL), | Gibco BRL | 15212012 |
| 10 mL | | |
| KaryoMAX ® Potassium Chloride Solution, | Gibco BRL | 10575090 |
| 0.075 M | | |
| PB-MAX ™ Karyotyping Medium (1X), | Gibco BRL | 12557021 |
| liquid | | |
| Portable Pipet-Aid device, rechargeable | VWR | 3498-103 |
| T25 culture flask with vent cap, | VWR | 89092-698 |
| non-treated, Corning | | |
| Benchtop Centrifuge, | VWR | 53513-800 |
| 4x 100 mL capacity | | |
| BD Falcon Centrifuge Tubes, | VWR | |
| conical, 15 mL | | |
| Serological Pipettes, Disposable, | VWR | |
| Plugged, 1 mL, 2 mL, 5 mL, 10 mL, 25 mL | | |
| VWR Superfrost Plus Micro slide, | VWR | 48311-703 |
| pack of 72 | | |
| Water bath, 37° C. | | |
| Incubator with 5% CO₂, 37° C. | | |

### Preparation

PB-MAX Karyotyping Medium (1×):

Thaw PB-MAX Karyotyping medium at 4° C. to 8° C. Warm the medium to room temperature and gently swirl to mix prior to use. PB-MAX Karyotyping medium can be thawed and aseptically transferred into smaller aliquots for convenience. These aliquots can be frozen and thawed at time of use, however multiple freeze-thaw cycles should be avoided. Avoid prolonged exposure to light when using this culture medium product.

Fixative:

Methanol:glacial acetic acid, 3:1. Prepare before each use.

KaryoMAX Potassium Chloride Solution, 0.075 M:

Prewarm the hypotonic solution to 37° C. prior to use.

### Procedure

Prepare mitotic cells from short-term blood cultures.

Add 10 mL of PB-MAX Karyotyping Medium to each sterile T-25 flask to be set up for the assay. (See Note 1)

Add 0.75 mL of heparinized blood to each T-25 flask.

Incubate for 72 hr at 37° C. (5% CO₂) in a cell culture incubator. Flasks should stand upright with caps loosely closed.

After 72 hr culture add 100 μl KaryoMAX Colcemid Solution (10 ug/mL) to each flask and mix well. Incubate for 30 min at 37° C.

After 30 minutes, transfer the culture to 15 mL centrifuge tubes and centrifuge at 1200 rpm for 10 min. Remove medium completely except for about 0.5 mL of supernatant remaining above the cell pellet.

Resuspend the cells gently in the remaining medium and carefully add approximately 2 mL of prewarmed (37° C.) KaryoMAX Potassium Chloride Solution, 0.075 M, drop-by-drop, while agitating gently. Add an additional 8 mL of KCl, for a total of 10 mL; mix well. (See Note below)

Incubate for 15 min at 37° C. in the water bath.

Add 0.5 mL of freshly prepared fixative, recap the tube, and invert to mix.

Centrifuge the cells at 1200 rpm for 5 minutes, and remove the supernatant.

Resuspend the cells and fix the cells by adding 10 mL of fixative; the first 2 mL should be added drop wise while agitating gently.

Incubate at for 10 minutes at room temperature, centrifuge the cells and remove the supernatant.

Repeat the fixation procedure two more times. It is not necessary to incubate the cells between centrifugations.

After the last centrifugation, resuspend the cells in 5.0 mL of fixative.

Store cell pellets in fixative at −20° C.

### Notes

White blood cells in peripheral blood must be stimulated with a mitogen, inducing cell division as a prerequisite for preparation of cells in metaphase. In preparations of peripheral human blood cells, T-lymphocytes are stimulated with phytohemagglutinin. PB-MAX Karyotyping Medium is composed of a liquid RPMI-1640 medium that is completely supplemented with standard concentrations of L-glutamine, gentamicin sulfate, fetal bovine serum and phytohemagglutinin. This formulation is based on Peripheral Blood Media referenced in ACT Laboratory manual (1991) for use in PHA-stimulated Peripheral Blood Culture.

Hypotonic treatment causes a swelling of the cells; the optimal time of treatment varies for different cell types and must be determined empirically.

### D. CEP (Chromosome Enumeration Probe) FISH Protocol

Labeled CEP (Chromosome Enumeration Probes) DNA probes can be used to identify human chromosomes in metaphase spreads and interphase nuclei with fluorescence in situ hybridization (FISH) for example to identify aneuploidies in normal and tumor cells, to serve as reference probe in cytogenetic studies and to identify the human chromosomes in hybrid cell lines. Table 13 shows reagents and instruments used.

### Specimen

Metaphase chromosomes and/or interphase nuclei of fixed cultured or uncultured cytological specimens prepared on microscope slides.

TABLE 13
Reagents and Instrumentation

| Item | Supplier | Catalog No. |
|---|---|---|
| Rainin Classic Starter Kit. 20/200/1000 | Rainin | PR-Start |
| μl Pipettes | | |
| Rainin PR-10, 0.5-10 uL | Rainin | PR-10 |
| Removable-cover racked tips 10 μl. | Rainin | RT-10S |
| Presterilized | | |
| Removable-cover racked tips 20 μl. | Rainin | RT-20S |
| Presterilized | | |
| Removable-cover racked tips 200 μl. | Rainin | RT-200S |
| Presterilized | | |
| Removable-cover racked tips 1000 μl. | Rainin | RT-1000S |
| Presterilized | | |
| Slide Warmer Space Saver, 120 V | VWR | 15160-795 |
| Analog Water Bath, 2.0 L 37° C. | VWR | 89032-196 |
| Analog Water Bath, 2.0 L 70° C. | VWR | 89032-196 |
| Microcentrigfuge Tubes (1.5 mL), | VWR | 20170-650 |
| natural, qty 250 | | |
| MiniFuge, 200 g, 6000 rpm, 120 V | VWR | 93000-196 |
| VWR Traceable Multi-colored Timer | VWR | 89087-400 |
| 60 mL (2.0 oz) glass coplin jar, case 6 | VWR | 25457-006 |
| Coplin Staining Jar, SCIENCEWARE, each | VWR | 47751-792 |
| VWR Cover Glass Forceps, straight | VWR | 82027-396 |
| VWR Slide Hybridization Oven, | VWR | 80087-000 |
| or 42° C. Incubator | | |
| Rubber Cement | VWR | 100491-938 |
| VWR Clear Bath, algicide, 8 0z. | VWR | 54847-540 |
| 20 x SSC, 1.0 L, DEPC treated | VWR | RLMB-045 |
| Ethanol Series 70%, 85%, 100% | | |
| Formamide, 500 mL | VWR | JTM520-7 |
| Kimwipes | | |
| CEP 4 SpectrumOrange Probe | Abbott | 06J36-014 |
| CEP 17 (D17Z1) SpectrumGreen Probe | Abbott | 06J37-027 |
| CEP Hybridization Buffer, 2 x 150 μL | Abbott | 07J36-001 |
| DAPI II Counterstain, 500 μL x 2 | Abbott | 06J50-001 |
| Antifade Solution, 240 μL x 2 | Abbott | 06J29-010 |
| Control low-level - female, 95% XY, 5% XX | Abbott | 07J21-011 |

Epifluoresence Microscope with filters and Imaging System

### Preparation

Note: Where indicated, measure the pH of these solutions at ambient temperature. Use a pH meter with a glass electrode unless otherwise noted.

2×SSC Solution:

Mix thoroughly 100 mL 20×SSC (pH 5.3) with 850 mL purified H₂O. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

Denaturation Solution (70% Formamide/2×SSC):

Mix thoroughly 49 mL ultrapure formamide, 7 mL 20×SSC (pH 5.3) and 14 mL purified H₂O in a glass coplin jar. Measure pH using pH indicator strips to verify pH is 7.0-8.0. Between uses, store covered at 2-8° C. Discard after 7 days. Prepare in glass coplin jar and heat to 73+/−1° C.

0.4×SSC/0.3% NP-40 Wash Solution:

Mix thoroughly 20 mL 20×SSC (pH 5.3) with 950 mL purified H₂O. Add 3 mL of NP-40. Mix thoroughly until NP-40 is completely dissolved. Measure pH and adjust pH to 7.0-7.5 with NaOH. Add purified H₂O to bring final volume of the solution to 1 liter. Store at ambient temperature. Discard stock solution after 6 months, or sooner if solution appears cloudy or contaminated. Prepare in glass coplin jar and heat to 73+/−1° C.

2×SSC/0.1% NP-40 Wash Solution:

Mix thoroughly 100 mL 20×SSC (pH 5.3) with 850 mL purified H₂O. Add 1 mL NP-40. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare in glass coplin jar and heat to 73+/−1° C.

Ethanol Solutions (70%, 85%, 100%):

Prepare v/v dilutions of 100% ethanol with purified H₂O. Between uses, store tightly covered at ambient temperature. Discard stock solutions after 6 months. Prepare 70%, 85% and 100% ethanol using distilled water in plastic coplin jars.

### Fluorescence In Situ Hybridization Procedure

### Probe Preparation

At room temperature mix 7 μL of CEP hybridization buffer, 1 μL CEP DNA probe, and 2 μL purified H₂O. Centrifuge for 1-3 seconds, vortex and then re-centrifuge.

Heat for 5 minutes in a 73° C. water bath, and then place on a slide warmer set to 45-50° C.

Vortex to mix. Spin the tubes briefly (1-3 seconds) in microcentrifuge to bring the contents to the bottom of the tube. Gently vortex again to mix.

### Denaturation of Specimen DNA (Control Slides or PHA-Stimulated Peripheral Blood Lymphocytes)

Prewarm the hybridization chamber (an airtight container) to 42° C. by placing it in the 42° C. incubator prior to slide preparation.

Add denaturing solution to Coplin jar and place in a 73±1° C. water bath for at least 30 minutes. Verify the solution temperature before use.

Denature the specimen DNA by immersing the prepared slides in the denaturing solution at 73±1° C. for 5 minutes. Do not denature more than 4 slides at one time per Coplin jar. Check that the pH of the denaturing solution is 7.0-8.0 before each use.

Using forceps remove the slide(s) from the denaturing solution and immediately place into a 70% ethanol wash solution at room temperature. Agitate the slide to remove the formamide. Allow the slide(s) to stand in the ethanol wash for 1 minute.

Remove the slide(s) from 70% ethanol. Repeat step 4 with 85% ethanol, followed by 100% ethanol.

Drain the excess ethanol from the slide by touching the bottom edge of the slide to a blotter and wipe the underside of the slide dry with a laboratory wipe.

Place the slide(s) on a 45-50° C. slide warmer no more than 2 minutes before you are ready to apply the probe solution.

Note: If the timing of the hybridization is such that the slide is ready more than 2 minutes before the probe is ready, the slide should remain in the jar of 100% ethanol. Do not air dry a slide before placing it on the slide warmer.

### Hybridization

Apply the 10 μL, aliquot of probe solution to the target area of the slide. Immediately, place a 22 mm×22 mm glass coverslip over the probe solution and allow the solution to spread evenly under the coverslip. Air bubbles will interfere with hybridization and should be avoided.

Note: Do not pipet probe solution onto multiple target areas before applying the coverslips.

Place the slide into the pre-warmed 42° C. hybridization chamber and cover the chamber with a tight lid.

Place the chamber containing the slide into the 42° C. incubator and allow hybridization to proceed for at least 30 minutes.

Note: Longer hybridization time may be required for sufficient signal intensity in some specimens. Incubations may be performed overnight (16 hours). For incubations longer than 1 hour, the coverslip must be sealed using a removable sealant such as rubber cement and the hybridization chamber must be humidified. The procedure is described below.

Draw rubber cement into a 5 mL syringe. Exude a small amount of rubber cement around the periphery of the coverslip overlapping the coverslip and the slide, thereby forming a seal around the coverslip.

Place the slide into a humidified hybridization chamber (an airtight container with a piece of damp blotting paper or paper towel approximately 1 in.×3 in. taped to the side of the container).

Cover the chamber with a tight lid and incubate 1 to 16 hours, as desired.

Following incubation, remove the rubber cement from the coverslip by pulling up on the rubber cement.

### Post-Hybridization Washes

Add 0.4×SSC (pH 7.0-7.5) to a Coplin jar. Prewarm the 0.4×SSC solution by placing the Coplin jar in the 73±1° C. water bath for at least 30 minutes or until the solution temperature has reached 73±1° C.

Note: In order to maintain the proper temperature range, four slides should be placed in the heated wash solution at one time. If fewer than four slides have been hybridized, room temperature microscope slides (without specimen applied) may be used to bring the number of slides to four. If more than four slides have been hybridized they must be washed in more than one batch. The temperature of the wash solution must return to 73±1° C. before washing each batch.

Remove the coverslip from the target area of the first slide and immediately place the slide into the Coplin jar containing 0.4×SSC, 73±1° C. Agitate the slide for 1-3 seconds. Repeat for the other three slides and incubate for 2 minutes at 73±1° C.

Note: Do not remove the coverslips from several slides before placing any of the slides in the wash bath. Begin timing the 2 minute incubation when the last slide has been added to the wash bath.

Remove each slide from the wash bath and place in the jar of 2×SSC/0.1% NP-40 at room temperature for 5-60 seconds, agitating for 1-3 seconds as the slides are placed in the bath.

Allow the slide to air dry in the dark. (A closed drawer or a shelf inside a closed cabinet is sufficient.)

Apply 10 μL of DAPI II counterstain to the target area of the slide and apply a glass coverslip. Store the slide(s) in the dark prior to signal enumeration.

### Storage

Store hybridized slides (with coverslips) at −20° C. in the dark. Under these conditions the slides can be stored for up to 12 months without significant loss in fluorescence signal intensity. For long-term storage, the coverslips should be sealed to prevent desiccation and the slides stored at −20° C.

### Signal Enumeration—Assessing Slide Adequacy

Evaluate slide adequacy using the following criteria:

Probe Signal Intensity: The signal should be bright, distinct, and easily evaluable. Signals should be in either bright, compact, oval shapes or stringy, diffuse, oval shapes.

Background: The background should appear dark or black and free of fluorescence particles or haziness.

Cross-hybridization/Target Specificity: The probe should hybridize and illuminate only the target (centromere of chromosome). Metaphase spreads should be evaluated to verify locus specificity and to identify any cross-hybridization to non-target sequences. At least 98% of cells should show one or more signals for acceptable hybridization.

### Signal Enumeration—Selection of Optimum Viewing Area and Evaluable Nuclei

Use a 25× objective to scan the hybridized area and examine the specimen distribution. Select an area where the specimen is distributed sparsely, few interphase nuclei are overlapping, and several interphase nuclei can be scanned within a viewing field. Avoid areas where the distribution of cells is dense, cells are overlapped, or the nuclear border of individual nuclei is unidentifiable. Avoid areas that contain clumps of cells. Enumerate only those cells with discrete signals.

### Signal Enumeration—Enumeration Scan

Using a 40× or 63× objective, begin analysis in the upper left quadrant of the selected area and, scanning from left to right, count the number of signals within the nuclear boundary of each evaluable interphase cell. Areas on the slide with a high cell density should be randomly skipped in order to scan the entire target area. Continue the scanning until 500 interphase nuclei are enumerated and analyzed.

### Signal Enumeration—Interphase Enumeration

Enumerate the fluorescent signals in each evaluable interphase nucleus using a 40× or 63× objective. Objectives with higher magnification (e.g., 63× or 100×) should be used to verify or resolve questions about split or diffused signals. Follow these guidelines:

Two signals that are in close proximity and approximately the same sizes but not connected by a visible link are counted as two signals.

Count a diffuse signal as one signal if diffusion of the signal is contiguous and within an acceptable boundary. Two small signals connected by a visible link are counted as one signal.

Enumerate the number of nuclei with 0, 1, 2, 3, 4, or >4 signals. Count nuclei with zero signals only if there are other nuclei with at least one signal present in the field of view. If the accuracy of enumeration is in doubt, repeat the enumeration in another area of the slide. Do not enumerate nuclei with uncertain signals.

### E. LSI (Locus Specific Identifier) FISH Protocol

The purpose of this protocol is to perform FISH using LSI (Locus Specific Identifier) probes on cytogenetic specimens. Labeled LSI DNA probes can be used to identify human chromosomes in metaphase spreads and interphase nuclei, and genetic aberrations with fluorescence in situ hybridization (FISH). For example the LSI BCR/ABL probe set is designed to detect fusion of the ABL gene locus on 9q34 and BCR gene locus on 22q11.2 (Translocation (9; 22)(q34; q11)). Table 14 shows reagents and instruments used.

### Specimen

Metaphase chromosomes and/or interphase nuclei of fixed cultured or uncultured cytological specimens prepared on microscope slides.

TABLE 14
Reagents and Instrumentation

| Item | Supplier | Catalog No. |
|---|---|---|
| Rainin Classic Starter Kit. 20/200/1000 | Rainin | PR-Start |
| μl Pipettes | | |
| Rainin PR-10, 0.5-10 uL | Rainin | PR-10 |
| Removable-cover racked tips 10 μl. | Rainin | RT-10S |
| Presterilized | | |
| Removable-cover racked tips 20 μl. | Rainin | RT-20S |
| Presterilized | | |
| Removable-cover racked tips 200 μl. | Rainin | RT-200S |
| Presterilized | | |
| Removable-cover racked tips 1000 μl. | Rainin | RT-1000S |
| Presterilized | | |
| Slide Warmer Space Saver, 120 V | VWR | 15160-795 |
| Analog Water Bath, 2.0 L 37° C. | VWR | 89032-196 |
| Analog Water Bath, 2.0 L 70° C. | VWR | 89032-196 |
| Microcentrigfuge Tubes (1.5 mL), | VWR | 20170-650 |
| natural, qty 250 | | |
| MiniFuge, 200 g, 6000 rpm, 120 V | VWR | 93000-196 |
| VWR Traceable Multi-colored Timer | VWR | 89087-400 |
| 60 mL (2.0 oz) glass coplin jar, case 6 | VWR | 25457-006 |
| Coplin Staining Jar, SCIENCEWARE, each | VWR | 47751-792 |
| VWR Cover Glass Forceps, straight | VWR | 82027-396 |
| VWR Slide Hybridization Oven, | VWR | 80087-000 |
| or 42° C. Incubator | | |
| Rubber Cement | VWR | 100491-938 |
| VWR Clear Bath, algicide, 8 0z. | VWR | 54847-540 |
| 20 x SSC, 1.0 L, DEPC treated | VWR | RLMB-045 |
| Ethanol Series 70%, 85%, 100% | | |
| Formamide, 500 mL | VWR | JTM520-7 |
| Kimwipes | | |
| Vysis LSI BCR/ABL Dual Color, Single Fusion | Abbott | 05J77-001 |
| Translocation Probe | | |
| LSI Hybridization Buffer, 2 x 150 μL | Abbott | 07J36-001 |
| DAPI II Counterstain, 500 μL x 2 | Abbott | 06J50-001 |
| Antifade Solution, 240 μL x 2 | Abbott | 06J29-010 |
| Control low-level - female, 95% XY, 5% XX | Abbott | 07J21-011 |

Epifluoresence Microscope with filters and Imaging System

### Preparation

Where indicated, measure the pH of these solutions at ambient temperature. Use a pH meter with a glass electrode unless otherwise noted.

2×SSC Solution:

Mix thoroughly 100 mL 20×SSC (pH 5.3) with 850 mL purified H₂O. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare 2×SSC in plastic coplin jar and preheat to 37° C. using a water bath.

Denaturation Solution (70% Formamide/2×SSC):

Mix thoroughly 49 mL ultrapure formamide, 7 mL 20×SSC (pH 5.3) and 14 mL purified H₂O in a glass coplin jar. Measure pH using pH indicator strips to verify pH is 7.0-8.0. Between uses, store covered at 2-8° C. Discard after 7 days. Prepare in glass coplin jar and heat to 73+/−1° C.

0.4×SSC/0.3% NP-40 Wash Solution:

Mix thoroughly 20 mL 20×SSC (pH 5.3) with 950 mL purified H₂O. Add 3 mL of NP-40. Mix thoroughly until NP-40 is completely dissolved. Measure pH and adjust pH to 7.0-7.5 with NaOH. Add purified H2O to bring final volume of the solution to 1 liter. Store at ambient temperature. Discard stock solution after 6 months, or sooner if solution appears cloudy or contaminated. Prepare in glass coplin jar and heat to 73+/−1° C.

2×SSC/0.1% NP-40 Wash Solution:

Mix thoroughly 100 mL 20×SSC (pH 5.3) with 850 mL purified H₂O. Add 1 mL NP-40. Measure pH and adjust to pH 7.0±0.2 with NaOH. Add purified H₂O to bring final volume to 1 liter. Store at ambient temperature. Discard stock solution after 6 months, or sooner if solution appears cloudy or contaminated. Prepare in glass coplin jar and heat to 73+/−1° C.

Ethanol Solutions (70%, 85%, 100%):

Prepare v/v dilutions of 100% ethanol with purified H₂O. Between uses, store tightly covered at ambient temperature. Discard stock solutions after 6 months. Prepare 70%, 85% and 100% ethanol using distilled water in plastic coplin jars.

### LSI Probe Preparation

At room temperature mix 7 ul of LSI Hybridization Buffer, 1 ul LSI DNA probe, and 2 ul purified H₂O. Centrifuge for 1-3 seconds, vortex and then re-centrifuge. Place on ice until use.

### Fluorescence In Situ Hybridization Procedure

Denaturation of Specimen DNA (Control Slides or PHA-Stimulated Peripheral Blood Lymphocytes)

Prewarm the hybridization chamber (an airtight container) to 37° C. by placing it in the 37° C. incubator prior to slide preparation.

Add denaturing solution to Coplin jar and place in a 73±1° C. water bath for at least 30 minutes. Verify the solution temperature before use.

Denature the specimen DNA by immersing the prepared slides in the denaturing solution at 73±1° C. for 5 minutes. Do not denature more than 4 slides at one time per Coplin jar. Check that the pH of the denaturing solution is 7.0-8.0 before each use.

Using forceps remove the slide(s) from the denaturing solution and immediately place into a 70% ethanol wash solution at room temperature. Agitate the slide to remove the formamide. Allow the slide(s) to stand in the ethanol wash for 1 minute.

Remove the slide(s) from 70% ethanol. Repeat step 4 with 85% ethanol, followed by 100% ethanol.

Drain the excess ethanol from the slide by touching the bottom edge of the slide to a blotter and wipe the underside of the slide dry with a laboratory wipe.

Place the slide(s) on a 45-50° C. slide warmer no more than 2 minutes before you are ready to apply the probe solution.

Note: If the timing of the hybridization is such that the slide is ready more than 2 minutes before the probe is ready, the slide should remain in the jar of 100% ethanol. Do not air dry a slide before placing it on the slide warmer.

### Probe Preparation

Heat the prepared probe for 5 minutes in a 73° C. water bath.

Place on a slide warmer set to 45-50° C. Cover tube with foil to block form light if not using right away.

### Hybridization

Apply the 10 μL aliquot of probe solution to the target area of the slide. Immediately, place a 22 mm×22 mm glass coverslip over the probe solution and allow the solution to spread evenly under the coverslip. Air bubbles will interfere with hybridization and should be avoided. Seal the coverslip with rubber cement.

Note: Do not pipet probe solution onto multiple target areas before applying the coverslips.

Place the slide into the pre-warmed 37° C. hybridization chamber and cover the chamber with a tight lid.

Place the chamber containing the slide into the 37° C. incubator and allow hybridization to proceed for 12-16 hours.

### Post-Hybridization Washes

Add 0.4×SSC (pH 7.0-7.5) to a Coplin jar. Prewarm the 0.4×SSC solution by placing the Coplin jar in the 73±1° C. water bath for at least 30 minutes or until the solution temperature has reached 73±1° C.

Note: In order to maintain the proper temperature range, four slides MUST be placed in the heated wash solution at one time. If fewer than four slides have been hybridized, room temperature microscope slides (without specimen applied) may be used to bring the number of slides to four. If more than four slides have been hybridized they must be washed in more than one batch. The temperature of the wash solution must return to 73±1° C. before washing each batch.

Remove the rubber cement and coverslip from the target area of the first slide and immediately place the slide into the Coplin jar containing 0.4×SSC, 73±1° C. Agitate the slide for 1-3 seconds. Repeat for the other three slides and incubate for 2 minutes at 73±1° C.

Note: Do not remove the coverslips from several slides before placing any of the slides in the wash bath. Begin timing the 2 minute incubation when the last slide has been added to the wash bath.

Remove each slide from the wash bath and place in the jar of 2×SSC/0.1% NP-40 at room temperature for 5-60 seconds, agitating for 1-3 seconds as the slides are placed in the bath.

Allow the slide to air dry in the dark. (A closed drawer or a shelf inside a closed cabinet is sufficient.)

Apply 10 μL of DAPI II counterstain to the target area of the slide and apply a glass coverslip. Store the slide(s) in the dark prior to signal analysis.

### Storage

Store hybridized slides (with coverslips) at −20° C. in the dark. Under these conditions the slides can be stored for up to 12 months without significant loss in fluorescence signal intensity. For long-term storage, the coverslips should be sealed to prevent desiccation and the slides stored at −20° C.

### Signal Analysis

### Assessing Slide Adequacy

The Triple bandpass filter DAPI/FITC/Texas Red is optimal for viewing all three fluorophores simultaneously. Evaluate slide adequacy using the following criteria:

Probe Signal Intensity: The signal should be bright, distinct, and easily evaluable. Signals should be in either bright, compact, oval shapes or stringy, diffuse, oval shapes.

Background: The background should appear dark or black and free of fluorescence particles or haziness.

Cross-hybridization/Target Specificity: The probe should hybridize and illuminate only the target. Metaphase spreads should be evaluated to verify locus specificity and to identify any cross-hybridization to non-target sequences.

### Selection of Optimum Viewing Area and Evaluable Nuclei

Use a 25× objective to scan the hybridized area and examine the specimen distribution. Select an area where the specimen is distributed sparsely, few interphase nuclei are overlapping, and several interphase nuclei can be scanned within a viewing field. Avoid areas where the distribution of cells is dense, cells are overlapped, or the nuclear border of individual nuclei is unidentifiable. Avoid areas that contain clumps of cells. Analyze only those cells with discrete signals.

### Interphase Enumeration

Analyze the fluorescent signals in each evaluable interphase nucleus using a 63× or 100× objective. In a normal cell, these probes will appear as discrete red (R) and green (G) spots, one for each homologue (resulting in a 2G 2R conformation). In a t(9:22) patient, there should be one yellow, white, or yellow-white (Y) fusion signal in addition to the red and green signals of the normal chromosome 9 and 22 respectively (1R 1G 1Y).

### Example 14

### Gene Expression Profile (GEP) Analysis

Gene expression profiles were generated for 1100 patients with Type2 Diabetes in clinical study (NucDia429), and 1100 patients with Type2 diabetes in clinical study (NucDia430). Expression data from the two studies were normalized together by Robust Microarray Analysis (RMA). Metrics associated with the two clinical study subsets are shown in Table 15.

TABLE 15
Comparison of two clinical study subsets

| | Study Identifier | Study Identifier |
|---|---|---|
| | (NucDia429) | (NucDia430) |
| Type 2 Diabetics | Obese glucose | Obese glucose |
| | intolerant group | intolerant group |
| Gene/Protein/Serum bio- | Yes | Yes |
| marker based determination | | |
| Patient Setting | Outpatient | Outpatient |
| Number of Patients | 1100 | 1100 |
| Collection Type | Sera | Sera |
| Gene array type | Affymetrix | Affymetrix |
| | HU133A - B | HU133A - B |

Gene expression data from the two studies was obtained via immunohistochemical methodology whereby serum biological samples were obtained from patients. Control samples were also obtained. Gene expression profiles (GEPs) then were generated from the samples based on total RNA according to well-established methods (See Affymetrix GeneChip expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix U133 GeneChip genomic array, and binding of the cDNA to the array was quantified by measuring the intensity of the signal from the detectable cDNA label bound to the array.

### Example 15

### FAS and Insulin Resistance

FASN values can be evaluated for insulin resistance based on the degree of regression since the degree of regression is a representation of insulin resistance. The regression analysis of the FASN data revealed that the relationship between FASN and insulin resistance is highly significant as the p-value for the likelihood ratio test and the Pearson's ChiSquare test is <0.001.

### Example 16

### Biomarkers for the Prediction of Insulin Resistance

Measurements of FASN, hemoglobin A1c (HBA1c) and glucose tolerance as biomarkers were evaluated based on clinical results to predict insulin resistance using the effect likelihood ratio test. The ChiSquare statistic for FASN was over 16 times larger than the next most significant variable. Glucose tolerance was also statistically significant (p=0.0167) whereas HBA1c was not significant (p=0.9345). The highest levels of FASN in serum were found in obese subjects who had altered glucose tolerance.

### Example 17

### Significance of Insulin Resistance Biomarkers

The significance of the insulin resistance biomarkers FASN, HBA1c, obesity body mass index (BMI) and glucose tolerance was evaluated using the effect likelihood ratio test and the ChiSquare test. The effect likelihood ratio test showed that only FASN was a significant biomarker having the largest ChiSquare test value. The results of the analysis are shown in Table 16.

TABLE 16
Significance of Insulin Resistance Biomarkers

| | Likelihood Ratio Test | | |
|---|---|---|---|
| Source | ChiSquare test value | P-value | Significance |
| HBA1c | 6.75 | 0.0243 | Not significant |
| Obesity BMI | 0.29 | 0.6937 | Not significant |
| FASN | 89.17 | <0.0001 | Significant |
| Glucose tolerance | 7.39 | 0.5329 | Not significant |

### Example 18

### Significance of Individual Biomarkers in Insulin Resistance

The individual biomarkers HBA1c, glucose tolerance, FASN, USP2a, BMI and NPY were evaluated by logistic regression and/or mosaic plot and the ChiSquare test against the degree of regression (DOR) (used as a representation of insulin resistance) to determine their significance on insulin resistance. The ChiSquare p-value showed that FASN and USP2a were a significant biomarker having the lowest p-value. The results of the analysis are shown in Table 17.

TABLE 17
Significance of Individual Biomarkers

| | ChiSquare p- | | |
|---|---|---|---|
| Comparison | value | Method of Analysis | Significance |
| DOR v. HBA1c | 0.7596 | Logistic Regression | Not significant |
| DOR v. HBA1c | 0.7156 | Mosaic Plot | Not significant |
| DOR v. Glucose | 0.3856 | Mosaic Plot | Not significant |
| Tolerance | | | |
| DOR v. FASN | <0.0001 | Mosaic Plot | Significant |
| DOR v. USP2a | <0.0001 | Mosaic Plot | Significant |
| DOR v. BMI | 0.3452 | Mosaic Plot | Not significant |
| DOR v. NPY | 0.4286 | Mosaic Plot | Not significant |

### Example 19

### Significance of Biomarkers in Insulin Resistance

The biomarkers HBA1c, glucose tolerance, FASN, USP2a, BMI and NPY were evaluated alone or in combination by logistic regression to determine the ChiSquare p-value to determine their significance on insulin resistance. The ChiSquare p-value showed that FASN alone and FASN with USP2a had the lowest p-value. The results of the analysis are shown in Table 18.

TABLE 18
Significance of Biomarkers

| | ChiSquare |
|---|---|
| Combination | p-value |
| FASN alone | <0.0001 |
| FASN and HBA1c | 0.2996 |
| FASN and USP2a | <0.0001 |
| FASN and USP2a and Glucose Tolerance | 0.0415 |
| FASN and BMI | 0.3952 |
| FASN and NPY | 0.0006 |
| USP2a and HBA1c | 0.0001 |

### Example 20

### Coefficient of Insulin Resistance Biomarkers

The coefficients of the biomarkers glucose tolerance, HBA1c, FASN and BMI were evaluated. Table 19 shows the estimated coefficient (Estimate), standard error (Std Error) the lower 95% confidence interval (Lower CL) and upper 95% confidence interval (Upper CL) in LogNormal distribution. The 95% confidence interval for the coefficient of HBA1c includes 0 which is an indication that HBA1c is not significant to insulin resistance prediction. The other 95% confidence intervals for glucose tolerance, FASN and BMI do not include zero so they may be significant. FASN and glucose tolerance have a negative coefficient which indicates that an increase in value is a predictor for insulin resistance. BMI has a positive coefficient indicating that longer therapy will have a better response to treatment.

TABLE 19
Coefficient Estimates of Model

| Biomarker | Estimate | Std Error | Lower CL | Upper CL |
|---|---|---|---|---|
| Glucose tolerance | −0.1589 | 0.0698 | −0.2714 | −0.0299 |
| HBA1c | −0.0011 | 0.0031 | −0.0079 | 0.0049 |
| FASN | −0.4729 | 0.0485 | −0.5102 | −0.4796 |
| BMI | 0.0330 | 0.0189 | 0.0089 | 0.0700 |

### Example 21

### Heart Failure Gene Expression Profile (GEP) Analysis

Gene expression profiles of blood samples were generated for 1068 patients in clinical studies CHF 0001 and CHF 0002. Metrics associated with the two clinical study subsets are shown in Table 20. The setting for both studies was inpatient treatment for heart failure.

Gene expression data from the two studies was obtained via gene array methodology utilizing the Affymetrix HU133A-B GeneChip® whereby blood samples were obtained from patients who had been diagnosed by a cardiologist or internist with either Stage B or Stage C HF. Blood samples from twenty healthy patients (free of cardiac disease) were used as negative controls and were simultaneously processed using the same techniques. The blood samples were subjected to density gradient centrifugation, and the DNA was extracted from the resulting buffy coat fraction using a commercial kit, such as the Qiagen® EZ1 DNA Blood kit and the EZ1 DNA Buffy Coat card.

Gene expression profiles (GEPs) then were generated from the biological samples based on total RNA according to well-established methods (See Affymetrix GeneChip® expression analysis technical manual, Affymetrix, Inc, Santa Clara, Calif.). Briefly, total RNA was isolated from the biological sample, amplified and cDNA synthesized. cDNA was then labeled with a detectable label, hybridized with a the Affymetrix HU133A-B GeneChip® genomic array, and binding of the cDNA to the array was quantified by measuring the intensity of the signal from the detectable cDNA label bound to the array.

TABLE 20
Comparison of two clinical study subsets

| | Study Identifier | Study Identifier |
|---|---|---|
| | (CHF 0001) | (CHF 0002) |
| Heart Failure Diagnosis | Stage B and C | Stage B and C |
| Number of patients: Total | 536 | 532 |
| Blood draw | Serum | Plasma |
| Identification of HF severity | 204 | 248 |
| Gene array type | Affymetrix | Affymetrix |
| | HU133A - B | HU133A - B |

*The term “severity” in Table 20 refers to the stage and stage progression of HF.

To develop a predictive GPEP (gene-protein expression profile), 37,452 probe sets were filtered by removing (a) probe sets with low expression over all samples; and (b) probe sets with low variance over all samples. This yielded 13,596 probe sets for subsequent analyses. Normalized log 2 (intensity) values were centered by subtracting the study-specific mean for each probe set, and rescaled by dividing by the pooled within-study standard deviation for each probe set.

A two-stage model-building approach was used to arrive at the best predictive model:

1. A single probe set analysis was used to search for probe sets that showed a difference between the two studies in the relationship between expression level and disease status, either by logistic regression or linear regression. This analysis yielded 586 probe sets.

2. A fit was analyzed with multi-probe-set predictive models. Here, the pre-selected probe sets from the single-probe-set analyses in step (1) were used as the starting point. Then the initial predictive models to each study were fit separately using a threshold gradient descent (TGD) method for regularized classification. Recursive feature elimination (RFE) was applied to attempt to simplify the models without appreciable loss of predictive accuracy.

The model selection criterion was the mean area under the ROC curve (AUC) from 50 replicates of a 4-fold cross-validation. Then from each RFE model series, here, one per study, the model with maximum difference between the selection criteria for the two studies was selected. The TGD method also was used to build predictive models based on expression of two individual probe sets.

Following the procedures outlined above, Signal-to-Noise ratios (S2N) were generated by comparing expression levels between Stage B and Stage C patients (the whole data set).

S2N was calculated based upon the following formula:

S2N=|x₁−x₂|/(s₁+s₂)

where xᵢ is the mean for trial i and sᵢ is the standard deviation for trial i, i=1,2.

Many microtubule-associated genes were identified with large S2N scores. GSTΩ1 (glutathione-S-transferase Ω1) had the largest ranking score and relatively wide expression range. Other genes with large signal-to-noise (S2N) scores among those with a range of at least 2.5 for log 2 (expression intensity) and P-value <0.01 for a t-test of the mean expression difference between Stage B and Stage C HF are shown in Table 21. Gene and Protein Reference Sequence refers to the sequence identifier of the gene from the NCBI database.

TABLE 21
Genes/Proteins having statistically significant signal-to-noise scores

| | | | Signal | | |
|---|---|---|---|---|---|
| | | Gene and | to | | |
| | | Protein | Noise | | SEQ |
| Gene | | Reference | score | P | ID |
| Symbol | Gene Name | Sequences* | (S/N) | value | NO |
| GSTΩ1 | Glutathione-S- | NM_004832.2 | 0.635 | 0.0004 | 1 |
| | transferase Ω 1 | | | | |
| SOD2 | Superoxide | NM_000636.2 | 0.884 | 0.0004 | 2 |
| | dismutase 2 | | | | |
| KCNE2 | potassium voltage- | NM_172201.1 | 0.901 | 0.0002 | 3 |
| | gated channel, | | | | |
| | Isk-related | | | | |
| | family, member 2 | | | | |
| BNP | Brain natriuretic | NM31776.1 | 0.935 | 0.0003 | 4 |
| | peptide | | | | |

*Gene sequence reference sequences have the “NM” prefix.

Table 21 sets forth a 4-gene profile or signature that is indicative of expression differences between patients having Stage B or C HF and normal healthy patients who were free of HF. This 4-gene GEP shows the top four differentially expressed genes in the pooled group of Stage B and C HF patients. All of the genes in the GEP were upregulated 2-fold to 4-fold in the HF patients who progressed to Stage C, compared to their levels in the healthy patients, and in those patients that remained stable in Stage B. The longest isoform of each gene is represented in Table 21; however, it is understood that other variants or isoforms of each gene may exist and that these are included within the embodiment of the gene.

Results of the analysis revealed the genes listed in Table 21 were identified as having the largest S2N scores and a relatively wide expression range.

Additional mRNA and protein sequences for the genes listed in Table 21 include those listed in Tables 22 and 23.

TABLE 22
mRNA variants

| Gene | | mRNA Reference | SEQ ID |
|---|---|---|---|
| Symbol | Gene Name | Sequences | NO |
| GSTΩ1 | Glutathione-S- | NM_001191003.1 | 5 |
| | transferase Ω 1 | NM_001191002.1 | 6 |
| SOD2 | Superoxide dismutase 2 | NM_001024466.1 | 7 |
| | | NM_001024465.1 | 8 |
| BNP | Brain natriuretic peptide | NM_002521.2 | 9 |

TABLE 23
Protein variants

| Gene | | Protein Reference | SEQ ID |
|---|---|---|---|
| Symbol | Gene Name | Sequences | NO |
| GSTΩ1 | Glutathione-S- | NP_001177932.1 | 10 |
| | transferase Ω 1 | NP_004823.1 | 11 |
| | | NP_001177931.1 | 12 |
| SOD2 | Superoxide dismutase 2 | NP_000627.2 | 13 |
| | | NP_001019637.1 | 14 |
| KCNE2 | potassium voltage-gated | NP_751951.1 | 15 |
| | channel, Isk-related | | |
| | family, member 2 | | |
| BNP | Brain natriuretic peptide | NP_002512.1 | 16 |

Given these findings, the present invention contemplates the use of at least two, at least 3 or at least 4 of the genes as a gene expression profile, the differential expression of which, either alone or in conjunction with imaging, will serve as a predictor of the likelihood of progression in individuals presenting with Stage B or C HF.

### Example 22

### Identification of GEP Subsets

The results of the analysis also identified two two-gene subsets that are indicative of the likelihood that patents with Stage B or C HF will worsen. These two two-gene GEPs are shown in Tables 24 and 25 respectively.

TABLE 24
Genes having statistically significant signal-to-noise scores (HF 1)

| | | | Signal | | |
|---|---|---|---|---|---|
| | | Gene and | to | | |
| | | Protein | Noise | | SEQ |
| Gene | | Reference | score | P | ID |
| Symbol | Gene Name | Sequences | (S/N) | value | NO |
| GSTΩ1 | Glutathione-S- | NM_004832.2 | 0.635 | 0.0004 | 1 |
| | transferase Ω 1 | | | | |
| SOD2 | Superoxide | NM_000636.2 | 0.884 | 0.0004 | 2 |
| | dismutase 2 | | | | |

TABLE 25
Genes having statistically significant signal-to-noise scores (HF 2)

| | | | Signal | | |
|---|---|---|---|---|---|
| | | Gene and | to | | |
| | | Protein | Noise | | SEQ |
| Gene | | Reference | score | P | ID |
| Symbol | Gene Name | Sequences | (S/N) | value | NO |
| KCNE2 | Potassium | NM_172201.1 | 0.901 | 0.0002 | 3 |
| | voltage-gated | | | | |
| | channel, Isk- | | | | |
| | related family, | | | | |
| | member 2 | | | | |
| BNP | Brain natriuretic | NM31776.1 | 0.935 | 0.0003 | 4 |
| | peptide | | | | |

The results of the expression analyses using the two 2-gene subsets are shown in Tables 26 and 27. These data illustrate that the two-marker model for both subsets (the presence of increased expression of these genes) predicted the likelihood that HF patients having Stage B or C HF would progress to a later stage with an accuracy of about 80-90% for signature 1 and about 90% for signature 2.

TABLE 26
Two-marker GEP predictive of progression of HF

| | | HF Stage B | | | HF Stage C | | |
|---|---|---|---|---|---|---|---|
| | | | | Detection | | | Detection |
| Model | Subset | R | N | Rate | R | N | Rate |
| GSTΩ1/ | All | 191 | 211 | 0.90 | 159 | 194 | 0.82 |
| SOD2 | patients | | | | | | |
| GSTΩ1 | CHF | 138 | 163 | 0.85 | 182 | 223 | 0.81 |
| SOD2 | CHF | 172 | 194 | 0.87 | 175 | 186 | 0.94 |

TABLE 27
Two-marker GEP predictive of progression of HF

| | | HF Stage B | | | HF Stage C | | |
|---|---|---|---|---|---|---|---|
| | | | | Detection | | | Detection |
| Model | Subset | R | N | Rate | R | N | Rate |
| KCNE2/ | All | 236 | 259 | 0.91 | 175 | 200 | 0.86 |
| BNP | patients | | | | | | |
| KCNE2 | CHF | 182 | 201 | 0.90 | 184 | 204 | 0.90 |
| BNP | CHF | 192 | 227 | 0.85 | 162 | 181 | 0.89 |

In Tables 26 and 27, R=True number of detections, N=Total number of patients in subset, Detection Rate=R/N. The detection rate for each condition for all patients, and for only patients with estimated detection probability was set at an arbitrary threshold of 0.5 based on expression level. The Detection Rate for Stage B means that the model detects Stage B stability, e.g., the probability that the HF patient in Stage B will remain in Stage B. None of the four genes were up-regulated in the Stage B patients whose disease was stable at Stage B, i.e., who did not progress to Stage C. All of these genes were upregulated in some of the Stage B patients and most of the Stage C patients. The Detection Rate for Stage C reflects the rate that patients move from Stage B to C, e.g., probability that the HF patient in Stage B will advance to Stage C.

Consequently, the studies provide two-marker GEPs where the level of expression may be employed as a tool, either alone or in conjunction with other GEPs or imaging techniques, to predict progression of HF to a later stage, in particular, from Stage B to Stage C.

### Example 23

### Gene Expression Profile (GEP) Analysis—Large Studies

Gene expression profiles of serum samples were generated for 2363 patients in clinical studies CHF 0003 and CHF 0004. Metrics associated with the two clinical study subsets are shown in Table 28. The setting for both studies was inpatient treatment for heart failure.

Gene expression data from the two studies was obtained via gene array methodology as described in Example 1 utilizing the Affymetrix HU133A-B GeneChip® whereby serum/plasma samples were obtained from patients who had been diagnosed by a cardiologist or internist with either Stage B (CHF 0003) or Stage C(CHF 0004) HF.

TABLE 28
Comparison of two clinical study subsets

| | Study Identifier | Study Identifier | |
|---|---|---|---|
| | (CHF 0003) | (CHF 0004) | Total Population |
| Heart Failure | Stage B | Stage C | Stage B + C |
| Diagnosis | | | |
| Number of patients: | 1166 | 1197 | 2363 |
| Total | | | |
| Blood draw | Serum, Plasma | Serum, Plasma | Serum, Plasma |
| Gene array type | Affymetrix | Affymetrix | Affymetrix |
| | HU133A - B | HU133A - B | HU133A - B |

A predictive GEP was developed using the two-stage approach described in Example 1. Following the procedures outlined in Example 1, Signal-to-Noise ratios (S2N) were generated by comparing expression levels between Stage B and Stage C patients (the whole data set). Twenty healthy patients (free of cardiac disease) were used as negative controls.

The results showing the mean expression difference between Stage B and Stage C HF are shown in Tables 29 and 30.

TABLE 29
Two-marker GEP predictive of progression of HF

| | | Stage B | | | Stage C | | |
|---|---|---|---|---|---|---|---|
| | | | | Detection | | | Detection |
| Model | Subset | R | N | Rate | R | N | Rate |
| None | All | — | 1166 | — | — | 1197 | — |
| | patients | | | | | | |
| GSTΩ1 | CHF | 974 | 1166 | 0.83 | 983 | 1197 | 0.82 |
| SOD2 | CHF | 962 | 1166 | 0.82 | 977 | 1197 | 0.81 |

TABLE 30
Two-marker GEP predictive of progression of HF

| | | Stage B | | | Stage C | | |
|---|---|---|---|---|---|---|---|
| | | | | Detection | | | Detection |
| Model | Subset | R | N | Rate | R | N | Rate |
| None | All | — | 1166 | — | — | 1197 | — |
| | patients | | | | | | |
| KCNE2 | CHF | 958 | 1166 | 0.82 | 963 | 1197 | 0.80 |
| BNP | CHF | 989 | 1166 | 0.85 | 969 | 1197 | 0.81 |

In Tables 29 and 30, R=True number of detections, N=Total number of patients in subset, Detection Rate=R/N. The detection rate for each condition for all patients, and for only patients with estimated detection probability was set at an arbitrary threshold of 0.5 based on expression level. The Detection Rate for Stage B reflects Stage B stability, e.g., the probability that the HF patient in Stage B will remain in Stage B. None of the four genes in Tables 29 and 30 are overexpressed in stable Stage B patients, whereas these genes are overexpressed in some of the Stage B patients and most of the Stage C patients. The Detection Rate for Stage C reflects the probability that the HF patient in Stage B will advance to Stage C.

The results in Tables 29 and 30 show that the detection rates of expression of these markers patients diagnosed with Stage C HF; these results indicate that Stage B HF patients that overexpress these markers are likely to progress to Stage C. Accordingly, the studies provide two-marker GEPs where the level of expression may be employed as a tool, either alone or in conjunction with other GEPs or imaging techniques, to predict progression of HF to a later stage.

The present invention contemplates the use of at least two, at least 3 or at least 4 of the genes as a gene expression profile, the differential expression of which, either alone or in conjunction with imaging, will serve as a predictor of the likelihood of progression in individuals presenting with Stage B or C HF.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. Where materials are incorporated by reference, all of the disclosure should be considered incorporated even when the incorporated material suggests a limitation to only the incorporated document or material. However, in case of conflict, the present specification, including definitions, will control.

In addition, section headings, the materials, methods, and examples are illustrative only and not intended to be limiting.

1. A method for predicting the incidence of metabolic syndrome in a subject comprising;
a. determining the level of one or more FASN SNPs selected from the group consisting of rs4246444, rs6502051 and rs12949488 in a sample obtained from the subject; and
b. stratifying the subject as likely to develop metabolic syndrome based on the level of said one or more FASN SNPs, wherein the level of said one or more FASN SNP demonstrates a greater than 0.7 signal to noise ratio.

2. The method of claim 1 wherein the subject has been diagnosed with type 2 diabetes.

3. The method of claim 2 wherein the subject is insulin resistant.

4. A method for predicting the incidence of metabolic syndrome in a subject comprising;
a. determining the level of expression of FASN or FASN in combination with USP2A in a sample obtained from the subject; and
b. stratifying the subject as likely to develop metabolic syndrome based on the expression level of FASN or FASN in combination with USP2A, wherein the detection rate of either FASN or FASN in combination with USP2A is independently 0.90 or greater.

5. The method of claim 4 wherein the subject has been diagnosed with type 2 diabetes.

6. The method of claim 5 wherein the subject is insulin resistant.

7. The method of claim 6 wherein protein expression levels are measured.

8. The method of claim 7 wherein protein levels are measured by an immunohistochemical assay.

9. The method of claim 8 wherein the immunohistochemical assay utilizes one or more FASN specific antibodies.

10. The method of claim 9, wherein said one or more FASN specific antibodies contains a detectable label.

11. An immunohistochemical kit or assay for the prediction or detection of metabolic syndrome comprising one or more FASN specific antibodies, each comprising a detectable label.

12. The immunohistochemical kit or assay of claim 11, further comprising a probe targeting the USP2a gene or protein.

13. The method of claim 1, further comprising measuring one or more clinical management parameter.

14. The method of claim 13, wherein said one or more clinical management parameters is selected from the group consisting of blood pressure, body mass index (BMI), levels of insulin, blood sugar, triglycerides, HDL, LDL and C-reactive protein.

15. A method for predicting the recurrence or aggressiveness of prostate cancer in a subject comprising;
a. determining the level of one or more SNPs selected from the group consisting of rs1447295, rs6983267, rs4430796, rs10993994, rs7127900, rs12621278, rs170021918, rs10486567, rs1512268, and rs12949488 in a sample obtained from the subject; and
b. stratifying the subject as likely to have a recurrence of cancer or an aggressive form of cancer based on the level of said one or more SNPs, wherein the level of said one or more SNPs is elevated over background.

16. A method for predicting the recurrence or aggressiveness of prostate cancer in a subject comprising;
a. determining the level of at least one SNP or expression product of one or more genes, the genes being selected from the group consisting of FTO (fat mass and obesity associated) gene, MC4R (melanocortin 4 receptor), TMEM18 (transmembrane protein 18), GNPDA2 (glucosamine-6-phosphate deaminase 2), ETV5 (Ets variant 5), BDNF (brain derived neurotrophic factor), SH2B1 (SH2B adapter protein 1), and PCSK1 (proprotein convertase subtilisin/kexin type 1) in a sample obtained from the subject; and
b. stratifying the subject as likely to have a recurrence of cancer or an aggressive form of cancer based on the level of said at least one SNP or expression product, wherein the level of said one or more SNPs or expression product is elevated over background.

17. The method of claim 16 wherein the expression product is a protein.

18. The method of claim 17 wherein protein levels are measured by an immunohistochemical assay.

19. The method of claim 18 wherein the immunohistochemical assay utilizes one or more specific antibodies.

20. A method of predicting whether a subject afflicted with early-stage heart failure will progress to a later stage comprising:
a. obtaining a biologic sample from the subject;
b. determining the level of one or more biomarker selected from the group consisting of FASN, FASN SNPs and USP2A; and
c. stratifying the subject as likely to progress to a later stage of heart failure based on the level of said one or more biomarker.

21. The method of claim 20, wherein the subject is afflicted with metabolic syndrome.

22. The method of claim 20 wherein the subject has been diagnosed with type 2 diabetes.

23. The method of claim 22 wherein the subject is insulin resistant.

24. A method for predicting whether a subject afflicted with metabolic syndrome will develop heart failure comprising:
a. obtaining a biologic sample from the subject;
b. determining the level of one or more biomarkers selected from the group consisting of FASN, USP2A, GSTΩ1, SOD2, KCNE2 and BNP in said biologic sample; and
c. stratifying the subject as likely to develop heart failure based on the expression level of said one or more biomarkers.

25. The method of claim 24 wherein the subject has been diagnosed with type 2 diabetes.

26. The method of claim 25 wherein the subject is insulin resistant.

27. The method of claim 24 wherein protein expression levels are measured.

28. A method of predicting the incidence of metabolic syndrome in a subject comprising;
a. obtaining a biologic sample from the subject; and
b. determining the expression level of one or more biomarkers in said biologic sample, wherein the biomarkers are selected from the group consisting of FASN, USP2A, GSTΩ1, SOD2, KCNE2 and BNP.

29. The method of claim 28 wherein the biologic sample obtained is selected from the group consisting of blood, peripheral blood mononuclear cells (PBMC), isolated blood cells, serum and plasma.

30. The method of claim 28 wherein protein expression levels are measured by immunoassay.

31. The method of claim 30 wherein the immunoassay method is an enzyme-linked immunosorbant assay (ELISA) method.

32. The method of claim 28 wherein the expression level of two biomarkers is determined.
